SEQUENCE LISTING (67 sequences)

SEQ ID NO: 1 - protein, 7 aa, Artificial Sequence
  Synthetic
PKKKRKV

SEQ ID NO: 2 - protein, 16 aa, Artificial Sequence
  Synthetic
KRPAATKKAGQAKKKK

SEQ ID NO: 3 - protein, 19 aa, Artificial Sequence
  Synthetic
KRTADGSEFESPKKKRKVE

SEQ ID NO: 4 - protein, 20 aa, Artificial Sequence
  Synthetic
AVKRPAATKKAGQAKKKKLD

SEQ ID NO: 5 - protein, 25 aa, Artificial Sequence
  Synthetic
MSRRRKANPTKLSENAKKLAKEVEN

SEQ ID NO: 6 - protein, 9 aa, Artificial Sequence
  Synthetic
PAAKRVKLD

SEQ ID NO: 7 - protein, 9 aa, Artificial Sequence
  Synthetic
KLKIKRPVK

SEQ ID NO: 8 - RNA, 35 nt, Artificial Sequence
  Synthetic
gucaaaagaccuuuuuaauuucuacucuuguagau

SEQ ID NO: 9 - RNA, 36 nt, Artificial Sequence
  Synthetic
guuucaaagauuaaauaauuucuacuaaguguagau

SEQ ID NO: 10 - RNA, 35 nt, Artificial Sequence
  Synthetic
gucuaacuaccuuuuaauuucuacuguuuguagau

SEQ ID NO: 11 - RNA, 36 nt, Artificial Sequence
  Synthetic
cucuagcaggccuggcaaauuucuacuguuguagau

SEQ ID NO: 12 - RNA, 43 nt, Artificial Sequence
  Synthetic
uaauuucuacucuuguagauucuguccccuccaccccacagug

SEQ ID NO: 13 - RNA, 44 nt, Artificial Sequence
  Synthetic
uaauuucuacuaaguguagauucuguccccuccaccccacagug

SEQ ID NO: 14 - RNA, 43 nt, Artificial Sequence
  Synthetic
uaauuucuacucuuguagauucaucugugccccucccucccug

SEQ ID NO: 15 - RNA, 44 nt, Artificial Sequence
  Synthetic
uaauuucuacuaaguguagauucaucugugccccucccucccug

SEQ ID NO: 16 - RNA, 43 nt, Artificial Sequence
  Synthetic
uaauuucuacucuuguagaucugaugguccaugucuguuacuc

SEQ ID NO: 17 - RNA, 44 nt, Artificial Sequence
  Synthetic
uaauuucuacuaaguguagaucugaugguccaugucuguuacuc

SEQ ID NO: 18 - RNA, 43 nt, Artificial Sequence
  Synthetic
uaauuucuacucuuguagaucuggagccugugauaaagcaacu

SEQ ID NO: 19 - DNA, 44 bp, Artificial Sequence
  Synthetic
uaauuucuacucuuguagauatcacaggctccaggaagggtttg

SEQ ID NO: 20 - RNA, 60 nt, Artificial Sequence
  Synthetic
cucuagcaggccuggcaaauuucuacuguuguagaucagucagggccauaggauagauau

SEQ ID NO: 21 - RNA, 59 nt, Artificial Sequence
  Synthetic
gucuaacuaccuuuuaauuucuacuguuuguagaucagucagggccauaggauagauau

SEQ ID NO: 22 - RNA, 59 nt, Artificial Sequence
  Synthetic
cucuagcaggccuggcaaauuucuacuguuguagauucaucugugccccucccucccug

SEQ ID NO: 23 - RNA, 59 nt, Artificial Sequence
  Synthetic
cucuagcaggccuggcaaauuucuacuguuguagauccaucaggcucucagcucagccu

SEQ ID NO: 24 - RNA, 59 nt, Artificial Sequence
  Synthetic
cucuagcaggccuggcaaauuucuacuguuguagauaggccccaguggcugcucugggg

SEQ ID NO: 25 - RNA, 58 nt, Artificial Sequence
  Synthetic
gucuaacuaccuuuuaauuucuacuguuuguagauucaucugugccccucccucccug

SEQ ID NO: 26 - RNA, 58 nt, Artificial Sequence
  Synthetic
gucuaacuaccuuuuaauuucuacuguuuguagauccaucaggcucucagcucagccu

SEQ ID NO: 27 - RNA, 58 nt, Artificial Sequence
  Synthetic
gucuaacuaccuuuuaauuucuacuguuuguagauaggccccaguggcugcucugggg

SEQ ID NO: 28 - DNA, 23 bp, Artificial Sequence
  Synthetic
aagatgaccagagcatccaaaag

SEQ ID NO: 29 - DNA, 23 bp, Artificial Sequence
  Synthetic
gtctgaatgccctctggatacag

SEQ ID NO: 30 - DNA, 20 bp, Artificial Sequence
  Synthetic
ctccaagcattgcatcatcc

SEQ ID NO: 31 - DNA, 19 bp, Artificial Sequence
  Synthetic
taccaggagatgcaatgct

SEQ ID NO: 32 - DNA, 23 bp, Artificial Sequence
  Synthetic
ccttcctggtaccaggatgatgc

SEQ ID NO: 33 - DNA, 23 bp, Artificial Sequence
  Synthetic
ctggtaccaggatgatgcaatgc

SEQ ID NO: 34 - DNA, 20 bp, Artificial Sequence
  Synthetic
ctaacagttgcttttatcac

SEQ ID NO: 35 - DNA, 23 bp, Artificial Sequence
  Synthetic
tctgtcccctccaccccacagtg

SEQ ID NO: 36 - RNA, 86 nt, Artificial Sequence
  Synthetic
  misc_feature (1)..(1): The proprietary AltR1 is at this position.
  misc_feature (86)..(86): The proprietary AltR2 is at this position.
rurararurururcrurarcrurcrururgrurargrarururcrurgrurcrcrcrcrurcrcrarcrcrcrcrarcrargrurg

SEQ ID NO: 37 - RNA, 86 nt, Artificial Sequence
  Synthetic
  misc_feature (1)..(1): The proprietary AltR1 is at this position.
  misc_feature (86)..(86): The proprietary AltR2 is at this position.
rurararurururcrurarcrurcrururgrurargrarururcrarurcrurgrurgrcrcrcrcrurcrcrcrurcrcrcrurg

SEQ ID NO: 38 - RNA, 86 nt, Artificial Sequence
  Synthetic
  misc_feature (1)..(1): The proprietary AltR1 is at this position.
  misc_feature (86)..(86): The proprietary AltR2 is at this position.
rurararurururcrurarcrurcrururgrurargrarurcrurgrarurgrgrurcrcrarurgrurcrurgrururarcrurc

SEQ ID NO: 39 - RNA, 88 nt, Artificial Sequence
  Synthetic
  misc_feature (1)..(1): The proprietary AltR1 is at this position.
  misc_feature (88)..(88): The proprietary AltR2 is at this position.
rurararurururcrurarcrurarargrurgrurargrarururcrurgrurcrcrcrcrurcrcrarcrcrcrcrarcrargrurg

SEQ ID NO: 40 - RNA, 88 nt, Artificial Sequence
  Synthetic
  misc_feature (1)..(1): The proprietary AltR1 is at this position.
  misc_feature (88)..(88): The proprietary AltR2 is at this position.
rurararurururcrurarcrurarargrurgrurargrarururcrarurcrurgrurgrcrcrcrcrurcrcrcrurcrcrcrurg

SEQ ID NO: 41 - RNA, 88 nt, Artificial Sequence
  Synthetic
  misc_feature (1)..(1): The proprietary AltR1 is at this position.
  misc_feature (88)..(88): The proprietary AltR2 is at this position.
rurararurururcrurarcrurarargrurgrurargrarurcrurgrarurgrgrurcrcrarurgrurcrurgrururarcrurc

SEQ ID NO: 42 - DNA, 49 bp, Artificial Sequence
  Synthetic
tctgggtactttatctgtcccctccaccccacagtggggccactaggga

SEQ ID NO: 43 - DNA, 50 bp, Artificial Sequence
  Synthetic
ggggcctcctgagtttctcatctgtgcccctccctccctggcccaggtga

SEQ ID NO: 44 - DNA, 50 bp, Artificial Sequence
  Synthetic
tctgggtacttttatctgtcccctccaccccacagtggggccactaggga

SEQ ID NO: 45 - DNA, 50 bp, Artificial Sequence
  Synthetic
cgttaatgtttcctgatggtccatgtctgttactcgcctgtcaagtggcg

SEQ ID NO: 46 - DNA, 44 bp, Artificial Sequence
  Synthetic
gccaaacccttcctggagcctgtgataaaagcaactgttagctt

SEQ ID NO: 47 - DNA, 27 bp, Artificial Sequence
  Synthetic
cttcctggagcctgtgataaaagcaac

SEQ ID NO: 48 - DNA, 27 bp, Artificial Sequence
  Synthetic
aaacccttcctggagcctgtgataaaa

SEQ ID NO: 49 - protein, 1365 aa, Artificial Sequence
  Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKGSYPYDVPDYAYPYDVPDYAYPYDVPDYAPKKKRKVHHHHHH

SEQ ID NO: 50 - protein, 1369 aa, Artificial Sequence
  Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKGSYPYDVPDYAYPYDVPDYAYPYDVPDYAPAAKRVKLDLEHHHHHH

SEQ ID NO: 51 - protein, 1384 aa, Artificial Sequence
  Synthetic
MPAAKRVKLDGGSGGGTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKGSYPYDVPDYAYPYDVPDYAYPYDVPDYAPAAKRVKLDLEHHHHHH

SEQ ID NO: 52 - protein, 1384 aa, Artificial Sequence
  Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKGSYPYDVPDYAYPYDVPDYAYPYDVPDYAPAAKRVKLDGGSGGGPAAKRVKLDLEHHHHHH

SEQ ID NO: 53 - protein, 1408 aa, Artificial Sequence
  Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKGSYPYDVPDYAYPYDVPDYAYPYDVPDYAPAAKRVKLDGGSGGGPAAKRVKLDLEGGGGKRTADSQHSTPPKTKRKVLEHHHHHH

SEQ ID NO: 54 - protein, 1286 aa, Artificial Sequence
  Synthetic
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYAPKKKRKVHHHHHH

SEQ ID NO: 55 - protein, 1290 aa, Artificial Sequence
  Synthetic
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYAPAAKRVKLDLEHHHHHH

SEQ ID NO: 56 - protein, 1306 aa, Artificial Sequence
  Synthetic
MPAAKRVKLDGGSGGGMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYAPAAKRVKLDLEHHHHHH

SEQ ID NO: 57 - protein, 1305 aa, Artificial Sequence
  Synthetic
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYAPAAKRVKLDGGSGGGPAAKRVKLDLEHHHHHH

SEQ ID NO: 58 - protein, 1329 aa, Artificial Sequence
  Synthetic
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYAPAAKRVKLDGGSGGGPAAKRVKLDLEGGGGKRTADSQHSTPPKTKRKVLEHHHHHH

SEQ ID NO: 59 - protein, 1384 aa, Artificial Sequence
  Synthetic
MPAAKRVKLDGGSGGGTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNREKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKGSYPYDVPDYAYPYDVPDYAYPYDVPDYAPAAKRVKLDLEHHHHHH

SEQ ID NO: 60 - protein, 1384 aa, Artificial Sequence
  Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNREKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKGSYPYDVPDYAYPYDVPDYAYPYDVPDYAPAAKRVKLDGGSGGGPAAKRVKLDLEHHHHHH

SEQ ID NO: 61 - protein, 1408 aa, Artificial Sequence
  Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNREKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKGSYPYDVPDYAYPYDVPDYAYPYDVPDYAPAAKRVKLDGGSGGGPAAKRVKLDLEGGGGKRTADSQHSTPPKTKRKVLEHHHHHH

SEQ ID NO: 62 - protein, 1328 aa, Artificial Sequence
  Synthetic
MPKKKRKVGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNRKRPAATKKAGQAKKKKGSYPYDVPDYAYPYDVPDYAYPYDVPDYAPAAKRVKLDLEHHHHHH

SEQ ID NO: 63 - protein, 1345 aa, Artificial Sequence
  Synthetic
MGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNRKRPAATKKAGQAKKKKGSYPYDVPDYAYPYDVPDYAYPYDVPDYAPAAKRVKLDGGSGGGKRTADSQHSTPPKTKRKVLEHHHHHH

SEQ ID NO: 64 - protein, 1338 aa, Artificial Sequence
  Synthetic
MPKKKRKVGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNRKRPAATKKAGQAKKKKGSYPYDVPDYAYPYDVPDYAYPYDVPDYAPAAKRVKLDLEHHHHHH

SEQ ID NO: 65 - protein, 1355 aa, Artificial Sequence
  Synthetic
MGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNRKRPAATKKAGQAKKKKGSYPYDVPDYAYPYDVPDYAYPYDVPDYAPAAKRVKLDGGSGGGKRTADSQHSTPPKTKRKVLEHHHHHH

SEQ ID NO: 66 - protein, 1335 aa, Artificial Sequence
  Synthetic
MPKKKRKVPKKKRKVGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNRKRPAATKKAGQAKKKKGSYPYDVPDYAYPYDVPDYAYPYDVPDYAPAAKRVKLDLEHHHHHH

SEQ ID NO: 67 - protein, 1352 aa, Artificial Sequence
  Synthetic
MPKKKRKVGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNRKRPAATKKAGQAKKKKGSYPYDVPDYAYPYDVPDYAYPYDVPDYAPAAKRVKLDGGSGGGKRTADSQHSTPPKTKRKVLEHHHHHH

(19) United States
(12) Patent Application Publication
(10) Pub. No.: US 2024/0287486 A1
(43) Pub. Date: Aug. 29, 2024

(54) IMPROVED CAS 12A/NLS MEDIATED THERAPEUTIC GENE EDITING PLATFORMS

(71) Applicant: UNIVERSITY OF MASSACHUSETTS, Boston, MA (US)

(72) Inventors: Scot A. Wolfe, Winchester, MA (US); Kevin Luk, Worcester, MA (US); PengPeng Liu, Worcester, MA (US)

(21) Appl. No.: 17/921,027

(22) PCT Filed: Apr. 21, 2021
(86) PCT No.: PCT/US21/28349

§ 371 Date: Oct. 24, 2022

Related U.S. Application Data
(60) Provisional application No. 63/014,986, filed on Apr. 24, 2020.

(51) Int. Cl. C12N 9/22; A61K 48/00; C12N 15/90

(52) U.S. Cl. CPC C12N 9/22; A61K 48/005; C12N 15/907; C07K 2319/09; C12N 2310/20; C12N 2800/80

(57) ABSTRACT

The present invention is related to the field of gene editing. In particular, the present invention is related to the mutation and/or deletion of genetic abnormalities that result in genetic diseases. For example, an improved CRISPR-Cas fusion protein is disclosed where the Cas protein is a Cas12a protein. The Cas12a protein is fused to a variety of nuclear localization signal (NLS) sequences (e.g., c-myc NLS) that are demonstrated to have unexpected and superior gene editing activity when compared to conventional NLS sequences (e.g., SV40 NLS).

### CROSS REFERENCE TO RELATED APPLICATIONS

This application claims priority to PCT/US21/28349, filed Apr. 21, 2021, now expired; which claims priority to Provisional Application Ser. No. 63/014,986 filed on Apr. 24, 2020, now expired, the contents of which are incorporated herein in their entirety.

### REFERENCE TO AN ELECTRONIC SEQUENCE LISTING

A Sequence Listing has been submitted in an ASCII text file named “19857_ST25.txt” created on Apr. 30, 2023, consisting of 235,224 bytes, the entire content of which is herein incorporated by reference.

### FIELD OF THE INVENTION

The present invention is related to the field of gene editing. In particular, the present invention is related to the mutation and/or deletion of genetic abnormalities that result in genetic diseases. For example, an improved CRISPR-Cas fusion protein is disclosed where the Cas protein is a Cas12a protein. The Cas12a protein is fused to a variety of nuclear localization signal (NLS) sequences (e.g., c-myc NLS) that are demonstrated to have unexpected and superior gene editing activity when compared to conventional NLS sequences (e.g., SV40 NLS).

### BACKGROUND

Cas9 (clustered regularly interspaced short palindromic repeats; CRISPR-associated system) may be part of a bacterial immune response to foreign nucleic acid introduction. The development of Type V CRISPR/Cas12 systems as programmable nucleases for genome engineering has been beneficial in the biomedical sciences. For example, a Cas9 platform has enabled gene editing in a large variety of biological systems, where both gene knockouts and tailor-made alterations are possible within complex genomes. The CRISPR/Cas12a system has the potential for application to gene therapy approaches for disease treatment, whether for the creation of custom, genome-edited cell-based therapies or for direct correction or ablation of aberrant genomic loci within patients.

The safe application of Cas12a in gene therapy requires exceptionally high precision to ensure that undesired collateral damage to the treated genome may be minimized or, ideally, eliminated. Numerous studies have outlined features of Cas12a that can drive editing promiscuity, and a number of strategies (e.g. truncated single-guide RNAs (sgRNAs), nickases and FokI fusions) have been developed that improve the precision of this system. However all of these systems still suffer from a degree of imprecision (cleavage resulting in lesions at unintended target sites within the genome).

However, what may be needed in the art are further improvements in Cas12a editing precision to facilitate reliable clinical applications that require simultaneous efficient and accurate editing of multigigabase genomes in billions to trillions of cells, depending on the scope of genetic repair that may be needed for therapeutic efficacy.

### SUMMARY

The present invention is related to the field of gene editing. In particular, the present invention is related to the mutation and/or deletion of genetic abnormalities that result in genetic diseases. For example, an improved CRISPR-Cas fusion protein is disclosed where the Cas protein is a Cas12a protein. The Cas12a protein is fused to a variety of nuclear localization signal (NLS) sequences (e.g., c-myc NLS) that are demonstrated to have unexpected and superior gene editing activity when compared to conventional NLS sequences (e.g., SV40 NLS).

In one embodiment, the present invention contemplates a Cas12a fusion protein comprising a Cas12a protein, at least one c-Myc nuclear localization sequence and a nucleoplasmin nuclear localization sequence. In one embodiment, a C-terminal portion of said Cas12a fusion protein comprises said at least one c-Myc nuclear localization signal sequence. In one embodiment, an N-terminal portion of said Cas12a fusion protein comprises said at least one c-Myc nuclear localization signal sequence. In one embodiment, said C-terminal portion of said Cas12a fusion protein comprises said nucleoplasmin nuclear localization signal sequence. In one embodiment, said Cas12a protein is selected from the group consisting of an Acidaminococcus sp Cas12a protein (AspCas12a or enAspCas12a), a Lachnospiraceae bacterium Cas12a protein (LbaCas12a), Moraxella bovoculi AAX08_00205 Cas12a protein (Mbo2Cas12a), Moraxella bovoculi AAX11_00205 Cas12a protein (Mbo3Cas12a) and Thiomicrospira sp Cas12a protein (TspCas12a). In one embodiment, the Cas12a fusion protein further comprises an SV40 nuclear localization signal sequence. In one embodiment, said SV40 nuclear localization signal sequence is a bipartite (BP) SV40 nuclear localization sequence. In one embodiment, said SV40 nuclear localization signal sequence is a large T antigen SV40 nuclear localization signal sequence. In one embodiment, the Cas12a fusion protein further comprises at least two c-Myc nuclear localization signal sequences. In one embodiment, a C-terminal portion of said Cas12a fusion protein comprises said at least two c-Myc nuclear localization signal sequences. In one embodiment, a N-terminal portion of said Cas12a fusion protein comprises said at least two c-Myc nuclear localization signal sequence. In one embodiment, the Cas12a fusion protein further comprises at least three c-Myc nuclear localization signal sequences. In one embodiment, a C-terminal portion of said Cas12a fusion protein comprises at least three c-Myc nuclear localization signal sequences and an N-terminal portion of said Cas12a fusion protein comprises at least one c-Myc nuclear localization signal sequences. In one embodiment, a C-terminal portion of said Cas12a fusion protein comprises said SV40 nuclear localization signal sequence. In one embodiment, a N-terminal portion of said Cas12a fusion protein comprises said SV40 nuclear localization signal sequence.

In one embodiment, the present invention contemplates a method, comprising: a) providing; i) a patient exhibiting at least one symptom of a genetic disease; ii) a pharmaceutically acceptable composition comprising a Cas12a fusion protein comprising a Cas12a protein, at least one c-Myc nuclear localization sequence and a nucleoplasmin nuclear localization sequence and a carrier; and b) administering said pharmaceutically acceptable composition to said patient under conditions such that said at least one symptom of said genetic disease is reduced. In one embodiment, said patient further comprises a mutated gene. In one embodiment, said administering further comprises gene editing wherein said mutated gene is deleted. In one embodiment, said administering further comprises gene editing wherein said mutated gene is inactivated. In one embodiment, said administering further comprises gene editing wherein said mutated gene is altered to restore function. In one embodiment, said administering further comprises gene editing wherein said mutated gene is converted to a wild type gene. In one embodiment, a C-terminal portion of said Cas12a fusion protein comprises said at least one c-Myc nuclear localization signal sequence. In one embodiment, an N-terminal portion of said Cas12a fusion protein comprises said at least one c-Myc nuclear localization signal sequence. In one embodiment, said C-terminal portion of said Cas12a fusion protein comprises said nucleoplasmin nuclear localization signal sequence. In one embodiment, said Cas12a protein is selected from the group consisting of an Acidaminococcus sp Cas12a protein (AspCas12a or enAspCas12a), a Lachnospiraceae bacterium Cas12a protein (LbaCas12a), Moraxella bovoculi AAX08_00205 Cas12a protein (Mbo2Cas12a), Moraxella bovoculi AAX11_00205 Cas12a protein (Mbo3Cas12a) and Thiomicrospira sp Cas12a protein (TspCas12a).

In one embodiment, the present invention contemplates a method, comprising: a) providing; i) a Cas12a fusion protein comprising a Cas12a protein, at least one c-Myc nuclear localization sequence and a nucleoplasmin nuclear localization sequence; and ii) at least one natural killer cell comprising at least one gene; b) transfecting the Cas12a fusion protien into the at least one natural killer cell, wherein the at least one gene is edited. In one embodiment, the at least one gene is an IFNG gene. In one embodiment, the at least one gene is a CD96 gene. In one embodiment, said gene is a mutated gene. In one embodiment, said edited gene is deleted. In one embodiment, said edited gene is inactivated. In one embodiment, said edited gene is altered to restore function. In one embodiment, said edited gene is converted to a wild type gene. In one embodiment, a C-terminal portion of said Cas12a fusion protein comprises said at least one c-Myc nuclear localization signal sequence. In one embodiment, an N-terminal portion of said Cas12a fusion protein comprises said at least one c-Myc nuclear localization signal sequence. In one embodiment, said C-terminal portion of said Cas12a fusion protein comprises said nucleoplasmin nuclear localization signal sequence. In one embodiment, said Cas12a protein is selected from the group consisting of an Acidaminococcus sp Cas12a protein (AspCas12a or enAspCas12a), a Lachnospiraceae bacterium Cas12a protein (LbaCas12a), Moraxella bovoculi AAX08_00205 Cas12a protein (Mbo2Cas12a), Moraxella bovoculi AAX11_00205 Cas12a protein (Mbo3Cas12a) and Thiomicrospira sp Cas12a protein (TspCas12a).

In one embodiment, the present invention contemplates a method, comprising: a) providing; i) a Cas12a fusion protein comprising a Cas12a protein, at least one c-Myc nuclear localization sequence and a nucleoplasmin nuclear localization sequence; and ii) at least one CD34⁺ hematopoietic stem and progenitor cell (HSPC) comprising at least one gene; b) transfecting the Cas12a fusion protien into the at least one HSPC, wherein the at least one gene is edited. In one embodiment, expression of a protein encoded by the edited gene is induced. In one embodiment, the protein is fetal γ-globin. In one embodiment, the at least one gene is a BCL11A gene. In one embodiment, said gene is a mutated gene. In one embodiment, said edited gene is deleted. In one embodiment, said edited gene is inactivated. In one embodiment, said edited gene is altered to restore function. In one embodiment, said edited gene is converted to a wild type gene. In one embodiment, a C-terminal portion of said Cas12a fusion protein comprises said at least one c-Myc nuclear localization signal sequence. In one embodiment, an N-terminal portion of said Cas12a fusion protein comprises said at least one c-Myc nuclear localization signal sequence. In one embodiment, said C-terminal portion of said Cas12a fusion protein comprises said nucleoplasmin nuclear localization signal sequence. In one embodiment, said Cas12a protein is selected from the group consisting of an Acidaminococcus sp Cas12a protein (AspCas12a or enAspCas12a), a Lachnospiraceae bacterium Cas12a protein (LbaCas12a), Moraxella bovoculi AAX08_00205 Cas12a protein (Mbo2Cas12a), Moraxella bovoculi AAX11_00205 Cas12a protein (Mbo3Cas12a) and Thiomicrospira sp Cas12a protein (TspCas12a).

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by the same series in reverse and then by 30 or so base pairs known as “spacer DNA”. The spacers are short segments of DNA from a virus and may serve as a ‘memory’ of past exposures to facilitate an adaptive defense against future invasions (PMID 25430774). These genomic segments are expressed as a precursor CRISPR RNAs (pre-crRNAs), which are then processed into mature crRNAs that program CRISPR effectors to their target sequence.

As used herein, the term “Cas” or “CRISPR-associated (cas)” refers to genes often associated with CRISPR repeat-spacer arrays (PMID 25430774).

As used herein, the term “Cas12a” refers to a nuclease from Type V CRISPR systems, an enzyme specialized for generating double-strand breaks in DNA, with a single active cutting domain (RuvC domain) that generates a double-strand break by breaking both strands of the double helix (Zetsche et al. Cell 2015 PMID 26422227). Cas12a protein are also known as Cpf1. Cas12a proteins can enzymatically process their own pre-crRNAs into crRNAs (Fonfara Nature 2016 PMID 27096362). When a crRNA is mixed with Cas12, it can find and cleave DNA targets through Watson-Crick pairing between the guide sequence within the crRNA and the target DNA sequence (Zetsche et al. Cell 2015 PMID 26422227). Cas12a systems do not require a tracrRNA for function. Common Cas12a systems include: Acidaminococcus sp Cas12a protein (AspCas12a; PMID 26422227), a Lachnospiraceae bacterium Cas12a protein (LbaCas12a; PMID 26422227), Francisella novicida U112 Cas12a protein (FnoCas12a; PMID 26422227), Moraxella bovoculi AAX08_00205 Cas12a protein (Mbo2Cas12a; PMID 31723075), Moraxella bovoculi AAX11_00205 Cas12a protein (Mbo3Cas12a; PMID 31723075) and Thiomicrospira sp Cas12a protein (TspCas12a; PMID 31723075). Engineered Cas12a variants have also been constructed with altered recognition specificity and editing activity (e.g. enAspCas12a; PMID 30742127).

As used herein, the term “Cas9” refers to a nuclease from Type II CRISPR systems, an enzyme specialized for generating double-strand breaks in DNA, with two active cutting sites (the HNH and RuvC domains), one for each strand of the double helix. Jinek combined tracrRNA and spacer RNA into a “single-guide RNA” (sgRNA) molecule that, mixed with Cas9, could find and cleave DNA targets through Watson-Crick pairing between the guide sequence within the sgRNA and the target DNA sequence (PMID 22745249).

As used herein, the term “nuclease deficient Cas9”, “nuclease dead Cas9” or “dCas9” refers to a modified Cas9 nuclease wherein the nuclease activity has been disabled by mutating residues in the RuvC and HNH catalytic domains. Disabling of both cleavage domains can convert Cas9 from a RNA-programmable nuclease into an RNA-programmable DNA recognition complex to deliver effector domains to specific target sequences (Qi, et al. 2013 (PMID 23452860) and Gilbert, et al. 2013 PMID 23849981) or to deliver an independent nuclease domain such as FokI. A nuclease dead Cas9 can bind to DNA via its PAM recognition sequence and guide RNA, but will not cleave the DNA.

The term “nuclease dead Cas9 FokI fusion” or “FokI-dCas9” as used herein, refers to a nuclease dead Cas9 that may be fused to the cleavage domain of FokI, such that DNA recognition may be mediated by dCas9 and the incorporated guide RNA, but that DNA cleavage may be mediated by the FokI domain (Tsai, et al. 2014 (PMID 24770325) and Guilinger, et al. (PMID 24770324)). FokI normally requires dimerization in order to cleave the DNA, and as a consequence two FokI-dCas9 complexes must bind in proximity in order to cleave the DNA. FokI can be engineer such that it functions as an obligate heterodimer.

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants that have either the RuvC or the HNH domain mutated provide control over which DNA strand is cleaved and which remains intact (Jinek, et al. 2012 (PMID 22745249) and Cong, et al. 2013 (PMID 23287718)).

The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into effector complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to base pairs with a pre-crRNA forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “programmable DNA binding domain” as used herein, refers to any protein comprising a pre-determined sequence of amino acids that bind to a specific nucleotide sequence. Such binding domains can include, but are not limited to, a zinc finger protein, a homeodomain and/or a transcription activator-like effector protein.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

As used herein, the term “sgRNA” refers to single guide RNA used in conjunction with CRISPR associated systems (Cas). sgRNAs are a fusion of crRNA and tracrRNA and contain nucleotides of sequence complementary to the desired target site (Jinek, et al. 2012 (PMID 22745249)). Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “orthogonal” refers targets that are non-overlapping, uncorrelated, or independent. For example, if two orthogonal Cas9 isoforms were utilized, they would employ orthogonal sgRNAs that only program one of the Cas9 isoforms for DNA recognition and cleavage (Esvelt, et al. 2013 (PMID 24076762)). For example, this would allow one Cas9 isoform (e.g. S. pyogenes Cas9 or spCas9) to function as a nuclease programmed by a sgRNA that may be specific to it, and another Cas9 isoform (e.g. N. meningitidis Cas9 or nmCas9) to operate as a nuclease dead Cas9 that provides DNA targeting to a binding site through its PAM specificity and orthogonal sgRNA. Other Cas9s include S. aureus Cas9 or SaCas9 and A. naeslundii Cas9 or AnCas9. Similarly orthogonal Cas12a proteins (e.g. AspCas12a and LbaCas12a) employ different crRNA sequences.

The term “truncated” as used herein, when used in reference to either a polynucleotide sequence or an amino acid sequence means that at least a portion of the wild type sequence may be absent. In some cases truncated guide sequences within the sgRNA or crRNA may improve the editing precision of Cas9 (Fu, et al. 2014 (PMID 24463574)).

The term “base pairs” as used herein, refer to specific nucleobases (also termed nitrogenous bases), that are the building blocks of nucleotide sequences that form a primary structure of both DNA and RNA. Double stranded DNA may be characterized by specific hydrogen bonding patterns, base pairs may include, but are not limited to, guanine-cytosine and adenine-thymine) base pairs.

The term “specific genomic target” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition sequence, an on-target binding sequence and an off-target binding sequence.

The term “on-target binding sequence” as used herein, refers to a subsequence of a specific genomic target that may be completely complementary to a programmable DNA binding domain and/or a single guide RNA sequence.

The term “off-target binding sequence” as used herein, refers to a subsequence of a specific genomic target that may be partially complementary to a programmable DNA binding domain and/or a single guide RNA sequence.

The term “fails to bind” as used herein, refers to any nucleotide-nucleotide interaction or a nucleotide-amino acid interaction that exhibits partial complementarity, but has insufficient complementarity for recognition to trigger the cleavage of the target site by the Cas9 nuclease. Such binding failure may result in weak or partial binding of two molecules such that an expected biological function (e.g., nuclease activity) fails.

The term “cleavage” as used herein, may be defined as the generation of a break in the DNA. This could be either a single-stranded break or a double-stranded break depending on the type of nuclease that may be employed.

As used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target or the specific inclusion of new sequence through the use of an exogenously supplied DNA template. Such a specific genomic target includes, but may be not limited to, a chromosomal region, mitochondrial DNA, a gene, a promoter, an open reading frame or any nucleic acid sequence.

As used herein, the term “hybridization” may be used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) may be impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

The term, “nuclear localization signal (NLS) sequence” as used herein refers to an amino acid sequence that ‘tags’ a protein for import into the cell nucleus by nuclear transport. Typically, this signal consists of one or more short sequences of positively charged lysines or arginines exposed on the protein surface. Different nuclear localized proteins may share the same NLS.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a sample, a compound or a sequence. In one respect, a sample, a compound or a sequence may be derived from an organism or particular species. In another respect, a sample, a compound or sequence may be derived from a larger complex or sequence.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that all trace impurities have been removed.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

The terms “amino acid sequence” and “polypeptide sequence” as used herein, are interchangeable and to refer to a sequence of amino acids.

The term “portion” when used in reference to a nucleotide sequence refers to fragments of that nucleotide sequence. The fragments may range in size from 5 nucleotide residues to the entire nucleotide sequence minus one nucleic acid residue. When used in reference to an amino acid sequence refers to fragments of that amino acid sequence. The fragment may range in size from 2 amino acid residues to the entire amino acid sequence minus one amino acid residue.

A “variant” of a protein is defined as an amino acid sequence which differs by one or more amino acids from a polypeptide sequence or any homolog of the polypeptide sequence. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions (i.e., additions), or both. Guidance in determining which and how many amino acid residues may be substituted, inserted or deleted without abolishing biological or immunological activity may be found using computer programs including, but not limited to, DNAStar® software.

A “variant” of a nucleotide is defined as a novel nucleotide sequence which differs from a reference oligonucleotide by having deletions, insertions and substitutions. These may be detected using a variety of methods (e.g., sequencing, hybridization assays etc.).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues, respectively, as compared to, for example, the naturally occurring amino acid sequence.

A “substitution” results from the replacement of one or more nucleotides or amino acids by different nucleotides or amino acids, respectively.

The term “derivative” as used herein, refers to any chemical modification of a nucleic acid or an amino acid. Illustrative of such modifications would be replacement of hydrogen by an alkyl, acyl, or amino group. For example, a nucleic acid derivative would encode a polypeptide which retains essential biological characteristics.

The term “biologically active” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells which lack protein activity with an expression vector which expresses the protein, a derivative thereof, or a portion thereof.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “bind” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

### BRIEF DESCRIPTION OF THE FIGURES

FIG. 1 presents exemplary data of optimized NLS architecture of Cas12a proteins.
FIG. 1A: Original design of 2×NLS Cas12a compared to AspCas12a and Lba Cas12a NLS variants. SV40=SV40 large-antigen NLS; NLP=Nucleoplasmin NLS; cMyc=cMyc NLS; 3×HA=3×Hemagglutinin Tag.
FIG. 1B: editing rates for different Cas12a RNP (defined in 1A) targeting DNMT1S3 and AAVS1S1in HEK293T cells.

FIG. 2 illustrates several embodiments of Cas12a fusion proteins;
i) prior art designs for 2×NLS-Nucleoplasmin (NLP)-SV40 Asp/LbaCas12a;
ii) 1×NLS-NLP enAspCas12a; and
iii) Asp/enAsp/Lba/Mbo2/Mbo3/TspCas12a NLS framework variants.

FIG. 3 presents exemplary data showing an assessment of editing efficiency by Cas12a RNP targeting AAVS1S1 sequence in HEK293T cells at 2.5 pmol and 5 pmol of protein:crRNA complex when delivered by nucleofection. Bars under named fusion protein indicate AspCas12a. Bars under named fusion protein indicate enAspCas12a. Bars under named fusion protein indicate LbaCas12a. Data is from three independent biological replicates. Error bars indicate±s.e.m. Statistical significance is determined by two-tailed Student's t-test: ns, P>0.05; *, P<0.01; **, P<0.001; ***, P<0.0001; ****, P<0.00001

FIG. 4 presents exemplary data showing an assessment of editing efficiency by Cas12a RNP targeting EMX1S1 in HEK293T cells at 2.5 pmol and 5 pmol of protein:crRNA complex when delivered by nucleofection. Bars under named fusion protein indicate AspCas12a. Bars under named fusion protein indicate enAspCas12a. Bars under named fusion protein indicate LbaCas12a. Data is from three independent biological replicates. Error bars indicate±s.e.m. Statistical significance is determined by two-tailed Student's t-test: ns, P>0.05; *, P<0.01; **, P<0.001; ***, P<0.0001; ****, P<0.00001.

FIG. 5 presents exemplary data showing an assessment of editing efficiency by rigorously purified Cas12a RNP targeting AAVS1S1 in HEK293T, Jurkat, and K562 cells at 5 pmol of protein:crRNA complex when delivered by nucleofection. Bars under named fusion protein indicate AspCas12a. Bars under named fusion protein indicate enAspCas12a. Bars under named fusion protein indicate LbaCas12a. Data is from three independent biological replicates. Error bars indicate±s.e.m. Statistical significance is determined by two-tailed Student's t-test: ns, P>0.05; *, P<0.01; **, P<0.001; ***, P<0.0001; ****, P<0.00001. 5pmol Cas12+12.5pmol crRNA.

FIG. 6 presents exemplary data showing an assessment of editing efficiency by rigorously purified Cas12a RNP targeting EMX1S1 in HEK293T, Jurkat, and K562 cells at 5 pmol of protein:crRNA complex when delivered by nucleofection. Bars under named fusion protein indicate AspCas12a. Bars under named fusion protein indicate enAspCas12a. Bars under named fusion protein indicate LbaCas12a. Data is from three independent biological replicates. Error bars indicate±s.e.m. Statistical significance is determined by two-tailed Student's t-test: ns, P>0.05; *, P<0.01; **, P<0.001; ***, P<0.0001; ****, P<0.00001. 5pmol Cas12+12.5pmol crRNA.

FIG. 7 presents exemplary data showing an assessment of editing efficiency by rigorously purified Cas12a RNP targeting DNMT1S3 in HEK293T, Jurkat, and K562 cells at 5 pmol of protein:crRNA complex when delivered by nucleofection. Bars under named fusion protein indicate AspCas12a. Bars under named fusion protein indicate enAspCas12a. Bars under named fusion protein indicate LbaCas12a. Data is from three independent biological replicates. Error bars indicate±s.e.m. Statistical significance is determined by two-tailed Student's t-test: ns, P>0.05; *, P<0.01; **, P<0.001; ***, P<0.0001; ****, P<0.00001. 5pmol Cas12+12.5pmol crRNA.

FIG. 8 presents exemplary data showing quantification of the nuclease activity of different NLS variants at different Cas12a RNP concentrations (20, 10, 5, and 2.5 pmol of protein:crRNA complex) at AAVS1S1 when delivered by nucleofection to HEK293T cells to determine optimal concentration to produce similar activities at the on-target site for specificity analysis. Bars under named fusion protein indicate AspCas12a. Bars under named fusion protein indicate enAspCas12a. Bars under named fusion protein indicate LbaCas12a.

FIG. 9 presents exemplary data showing quantification of the nuclease activity of different NLS variants at different Cas12a RNP concentrations (20, 10, 5, and 2.5 pmol of protein:crRNA complex) at DNMT1S3 when delivered by nucleofection to HEK293T cells to determine optimal concentration to produce similar activities at the on-target site for specificity analysis. Bars under named fusion protein indicate AspCas12a. Bars under named fusion protein indicate enAspCas12a. Bars under named fusion protein indicate LbaCas12a.

FIG. 10 presents exemplary data showing gene editing activity of various Cas12a nucleases, where NLS configurations are: 1=2×NLS-NLP-SV40 Cas12a; 2=2×NLS-NLP-cMyc Cas12a; 3=3×NLS-NLP-cMyc-cMyc Cas12a, where line bars indicate AspCas12a, white bars indicate enAspCas12a, spotted bars indicate LbaCas12a. Bars indicate editing rates at the on-target site and 13 GUIDE-seq and computationally identified potential off-target sites (OT1-OT13) for crRNA targeting DNMT1S3. The Cas12a proteins were delivered at specific concentrations of protein:crRNA complex (2×NLS-NLP-SV40 AspCas12a: 5 pmol; 2×NLS-NLP-cMyc AspCas12a: 5 pmol; 3×NLS-NLP-cMyc-cMyc AspCas12a: 2.5 pmol; 3×NLS-NLP-cMyc-cMyc enAspCas12a: 1.25 pmol; 2×NLS-NLP-SV40 LbaCas12a: 40 pmol; 2×NLS-NLP-cMyc LbaCas12a: 40 pmol; 3×NLS-NLP-cMyc-cMyc Cas12a: 40 pmol) to achieve approximately 80% editing at the target site. Data is from three independent biological replicates determined by Illumina deep sequencing. Error bars indicate±s.e.m.

FIG. 11 presents one embodiment of a nucleotide sequence surrounding 1617 sgRNA target site in the +58 element of BCL11A. Bracketed is the GATA1 binding motif. Boxes indicate the PAM sequence and arrows indicate the protospacer sequence with the direction indicating 5′->3′ (right to left arrows indicate protospacers on the complementary strand). The guides for enAspCas12a target sites 1 (TS1, left) and 2 (TS2, right) are indicated.

FIG. 12 shows exemplary data showing the quantification of editing efficiency of single replicate experiment using enAspCas12a with crRNAs targeting two different sites as described in FIG. 11 in the +58 element of BCL11A for 20 pmol 3×NLS-NLP-cMyc-cMyc enAsCas12a RNPs delivered by nucleofection to HEK293T cells.

FIG. 13 shows exemplary data showing an indel spectrum produced by enAspCas12a at two different sites in the +58 element of BCL11a (TS1 and TS2) demonstrates efficient disruption of the GATA1-binding motif.

FIG. 14 presents exemplary data of an in vitro cleavage assay at three different EMX1 target sites bearing three different PAM sequences (TTTC, CTTC, and GTTG). Upper arrow indicates the uncut DNA amplicon and black brackets indicate the cleaved DNA product, which vary in length depending on the position of the Cas12a target site within the PCR amplicon. Image of agarose gel electrophoresis of DNA after treatment with the indicated Cas12a-crRNA combinations (g1=TS1; g2=TS2; g3=TS3).

FIG. 15 presents exemplary data showing quantification of editing efficiency of single replicate experiment using Mbo2Cas12a, Mbo3Cas12a, and TspCas12a with crRNAs targeting three different EMX1 target sites bearing three different PAM sequences (TTTC (TS1), CTTC (TS2), and GTTG (TS3)) with 10 pmol of protein:crRNA complex in HEK293T cells.

FIG. 16 presents an exemplary 2×NLS-NLP-SV40 AspCas12a (Old-2C-AspCas12a) construct schematic and associated protein sequence.

FIG. 17 presents an exemplary 2×NLS-NLP-cMyc AspCas12a (NEW-2C-AspCas12a) construct schematic and associated protein sequence.

FIG. 18 presents an exemplary 1N/2CxNLS-cMyc-NLP-cMyc AspCas12a (1N-2C-AspCas12a) construct schematic and associated protein sequence.

FIG. 19 presents an exemplary 3×NLS-NLP-cMyc-cMyc AspCas12a (3C-AspCas12a) construct schematic and associated protein sequence.

FIG. 20 presents an exemplary 4×NLS-NLP-cMyc-cMyc-BPSV40 AspCas12a (4C-AspCas12a) construct schematic and associated protein sequence.

FIG. 21 presents an exemplary 2×NLS-NLP-SV40 LbaCas12a (Old-2C-LbaCas12a) (PMID 30892626; and PMID: 30704988) construct schematic and associated protein sequence.

FIG. 22 presents an exemplary 2×NLS-NLP-cMyc LbaCas12a (NEW-2C-LbaCas12a) construct schematic and associated protein sequence.

FIG. 23 presents an exemplary 1N/2CxNLS-cMyc-NLP-cMyc LbaCas12a (1N-2C-LbaCas12a) construct schematic and associated protein sequence.

FIG. 24 presents an exemplary 3×NLS-NLP-cMyc-cMyc LbaCas12a (3C-LbaCas12a) construct schematic and associated protein sequence.

FIG. 25 presents an exemplary 4×NLS-NLP-cMyc-cMyc-BPSV40 LbaCas12a (4C-LbaCas12a) construct schematic and associated protein sequence.

FIG. 26 presents an exemplary 1N/2CxNLS-cMyc-NLP-cMyc enAspCas12a (1N-2C-enAspCas12a) construct schematic and associated protein sequence.

FIG. 27 presents an exemplary 3×NLS-NLP-cMyc-cMyc enAspCas12a (3C-enAspCas12a): construct schematic and associated protein sequence.

FIG. 28 presents an exemplary 4×NLS-NLP-cMyc-cMyc-BPSV40 enAspCas12a (4C-enAspCas12a) construct schematic and associated protein sequence.

FIG. 29 presents an exemplary 1N/2CxNLS-SV40-NLP-cMyc Mbo2Cas12a (1N-2C-Mbo2Cas12a) construct schematic and associated protein sequence.

FIG. 30 presents an exemplary 3×NLS-NLP-cMyc-BPSV40 Mbo2Cas12a (3C-Mbo2Cas12a) construct schematic and associated protein sequence.

FIG. 31 presents an exemplary 1N/2CxNLS-SV40-NLP-cMyc Mbo3Cas12a (1N-2C-Mbo3Cas12a) construct schematic and associated protein sequence.

FIG. 32 presents an exemplary 3×NLS-NLP-cMyc-BPSV40 Mbo3Cas12a (3C-Mbo3Cas12a) construct schematic and associated protein sequence.

FIG. 33 presents an exemplary 1N/2CxNLS-SV40-NLP-cMyc TspCas12a (1N-2C-TspCas12a) construct schematic and associated protein sequence.

FIG. 34 presents an exemplary 3×NLS-NLP-cMyc-BPSV40 TspCas12a (3C-TspCas12a) construct schematic and associated protein sequence.

FIG. 35 presents exemplary target site editing data in the form of TIDE analysis (PMID 25300484) of Sanger sequencing of a population of edited cells by 3×NLS-NLP-cMyc-cMyc LbaCas12a (3C-LbaCas12a) with guide 1 delivered as a protein: RNA complex by electroporation to B-EBV cells containing a GATA microduplication in the HexA locus (GM11852 TAY-SACHS DISEASE; Coriell).

FIG. 36 presents exemplary target site editing data in the form of TIDE analysis (PMID 25300484) of Sanger sequencing of a population of edited cells by 3×NLS-NLP-cMyc-cMyc LbaCas12a (3C-LbaCas12a) with guide 2 delivered as a protein: RNA complex by electroporation to B-EBV cells containing a GATA microduplication in the HexA locus (GM11852 TAY-SACHS DISEASE; Coriell).

FIG. 37 presents exemplary target site editing data in the form of TIDE analysis (PMID 25300484) of Sanger sequencing of a population of edited cells by 3×NLS-NLP-cMyc-cMyc enAspCas12a (3C-enAspCas12a) with guide 1 delivered as a protein: RNA complex by electroporation to B-EBV cells containing a GATA microduplication in the HexA locus (GM11852 TAY-SACHS DISEASE; Coriell).

FIG. 38 presents exemplary target site editing data in the form of TIDE analysis (PMID 25300484) of Sanger sequencing of a population of edited cells by 3×NLS-NLP-cMyc-cMyc LbaCas12a (3C-LbaCas12a) with guide 1 delivered as a protein: RNA complex by electroporation to B-EBV cells containing a wild-type sequence in the HexA locus.

FIG. 39 presents exemplary target site editing data in the form of TIDE analysis (PMID 25300484) of Sanger sequencing of a population of edited cells by 3×NLS-NLP-cMyc-cMyc LbaCas12a (3C-LbaCas12a) with guide 2 delivered as a protein: RNA complex by electroporation to B-EBV cells containing a wild-type sequence in the HexA locus.

FIG. 40 presents exemplary target site editing data in the form of TIDE analysis (PMID 25300484) of Sanger sequencing of a population of edited cells by 3×NLS-NLP-cMyc-cMyc enAspCas12a (3C-enAspCas12a) with guide 1 delivered as a protein: RNA complex by electroporation to B-EBV cells containing a wild-type sequence in the HexA locus.

FIG. 41 presents exemplary data to assess 1×NLS enAspCas12a or 3×NLS enAspCas12a nuclease activity on the IFNG gene at 5 days post-nucleofection in NK cells subsequent to stimulation with an PMA+ionomycin or a IL-12+IL-15+IL-18 cocktail for 3 hours or overnight. NK cells were electroporated with 2 doses (50pmol or 200pmol RNP).
FIG. 41A: Indel Rate (%).
FIG. 41B: % Knockout as measured by loss of IFNG expression. % Knockout=% IFNG+ cells in experimental group/% IFNG+ cells of non-target group.

FIG. 42 presents exemplary data to assess 1×NLS enAspCas12a or 3×NLS enAspCas12a nuclease activity on the CD96 gene at 5 days post-nucleofection in NK cells subsequent to stimulation with an PMA+ionomycin or a IL-12+IL-15+IL-18 cocktail for 3 hours or overnight. NK cells were electroporated with 2 doses (50pmol or 200pmol RNP).
FIG. 42A: Indel Rate (%).
FIG. 42B: % Knockout as measured by loss of CD96 expression. % Knockout=% CD96+ cells in experimental group/% CD96+ cells of non-target group.

FIG. 43 presents exemplary data showing a representative flow cytometry plot on the cells expressing IFNy or CD96. Cells were stained with anti-Lin and anti-CD56 antibodies to gate for NK cells and then stained with anti-IFNy and anti-CD96 antibodies for expression of proteins. Box represent gate for populations of interest. Non-target control is an RNP targeting DNMT1S3.

FIG. 44 presents one embodiment of a sequence surrounding ATF4-binding motif in the +55 enhancer of Bcl11a and GATA1-binding motif in the +58 enhancer of Bcl11a. Each target site is highlighted by rectangular box. Pointed ends of boxes indicate the directionality of the guide RNA (5′-3′), where the right to left arrows indicate protospacers on the complementary strand. 1617=sgRNA targeting the GATA1 binding motif in the +58 enhancer of BCL11a. Specific binding motifs of interest are outlined by a box. Triangles indicate the potential positions of double-stranded breaks induced by the targets. ATF4 and GATA1 binding sites are outlined by a box. Triangles indicate position of DSBs.

FIG. 45 presents exemplary data showing a quantification of the nuclease activity at different target sites with respective Cas nucleases at varying RNP concentrations (100, 50, 25, and 21.5 pmol of protein:crRNA complex) when delivered by nucleofection to HEK293T cells to determine target site and nucleases of interest for CD34+ hematopoietic stem and progenitor cells (HSPCs) experiments.

FIG. 46 presents exemplary data showing a quantification of editing efficiency at 3 days post-electroporation and levels of HbF induction in differentiated erythrocytes (18 days post-electroporation) in two separate donors (Donor 1 and Donor 2). Target sites and nucleases validated in HEK293T experiments were utilized in this experiment. AAVS1S1 serves as a non-target control for enAspCas12a. HbF induction is quantified via HPLC. SpyCas9 TS2 and MboCas12a ATF4 TS2 are not further evaluated due to either lack on nuclease activity and/or lack of levels of HbF induction in comparison to other groups.
FIG. 46A: Indel rate in Donor 1 and Donor 2.
FIG. 46B: HbF induction in Donor 1.
FIG. 46C: HbF induction in Donor 2.

FIG. 47 presents exemplary data that compares editing efficiency at 3 days post-electroporation and levels of HbF induction in differentiated erythrocytes (18 days post-electroporation) of SpyCas9 1617, SpyCas9 ATF4 TS1, and 3×NLS enAspCas12a TS2 in multiple donors. Each donor is represented by specific colored shape as noted in the legend. Dashed lines indicated grant mean of the group.
FIG. 47A: Indel rate.
FIG. 47B. HbF induction.

FIG. 48 presents exemplary data that compares editing efficiency at 3 days post-electroporation and levels of HbF induction in differentiated erythrocytes (18 days post-electroporation) of 1×NLS enAspCas12a-HF1 and 3×NLS enAspCas12a-HF1 at TS2 in multiple donors. HF1 denotes the high-fidelity version of the Cas12a nuclease. Each donor is represented by specific colored shape as noted in the legend. Dashed lines indicated grant mean of the group.
FIG. 48A: Indel rate.
FIG. 48B. HbF induction.

### DETAILED DESCRIPTION OF THE INVENTION

The present invention is related to the field of gene editing. In particular, the present invention is related to the mutation and/or deletion and/or correction of genetic abnormalities that result in genetic diseases. For example, an improved CRISPR-Cas fusion protein is disclosed where the Cas protein is a Cas12a protein. The Cas12a protein is fused to a variety of nuclear localization signal (NLS) sequences (e.g., c-myc NLS) that are demonstrated to have unexpected and superior gene editing activity when compared to conventional NLS sequences (e.g., SV40 NLS).

### I. Type V CRISPR-Cas12a Systems

Type V CRISPR-Cas12a nucleases are a well-characterized gene-editing platform that has been used for editing in vertebrate and non-vertebrate systems. Reports have shown that SpyCas9 and Cas12a fused with different NLS frameworks result in more robust genome editing platforms in transformed mammalian cells lines, CD34+ HSPCs, and zebrafish. However, the efficiency of mutagenesis by Cas12a is lower compared to CRISPR-Cas9, especially in quiescent primary cells. Furthermore, Cas12a nucleases are not as widely utilized in the therapeutic community.

Herein, modifications to Cas12a nucleases are described that improve indel rates in transformed mammalian cells lines and quiescent primary cells. Similar to previous reports described for SpyCas9, is was found that the number and composition of the NLSs significantly impact the nuclease activity of Cas12a. More specifically, substitution of the previously utilized SV40 T antigen NLS for the more efficient, c-Myc NLS, and addition of a third NLS to the C-terminal of Cas12a resulted in a more robust genome editing platform. This enhancement of activity was irrespective of Cas12a ortholog (Asp, enAsp, or Lba).

Clustered regularly interspaced short palindromic repeats (CRISPR)-Cas12a is a type V CRISPR-Cas system that has been well characterized and harnessed by the research community for genome editing (e.g., PMID: 26422227 and PMID: 26593719). This Cas12a system provides an attractive alternative nuclease platform for specific genome editing applications to the Cas9 system that is being broadly utilized within the research community and for the development of therapeutics.

There are several unique characteristics that distinguish Cas12a from the more commonly utilized Cas9 nuclease platform from Streptococcus pyogenes (SpyCas9). First, the most commonly employed Cas12a nucleases from Acidaminococcus sp (Asp) and Lachnospiraceae bacterium (Lba) recognize a T-rich (TTTV [V=A/G/C]) protospacer adjacent motif (PAM) sequence and utilize a single ˜42 nucleotide CRISPR RNA (crRNA) for target site recognition (e.g., PMID: 26422227 and PMID: 27992409). Additionally, Cas12a cuts distally from its PAM sequence in a staggered fashion, generating a double strand break (DSB) with four or five nucleotide 5′-overhangs, whereas Cas9 cuts proximal to its PAM, typically generating a blunt ended DSB (e.g., PMID: 26422227 and PMID: 27096362). These properties, along with the favorable precision of Cas12a nuclease platforms (e.g., PMID: 27272384; PMID: 27347757; and PMID: 28497783), provides potential advantages of Cas12a over Cas9-based systems for therapeutic applications.

AspCas12a and LbaCas12a are believed to be the most widely employed Cas12a nucleases in the genome editing field due to demonstrated activity in a number of biological systems including fruit flies (e.g., PMID: 27595403), mammalian cells (e.g., PMID: 26422227; PMID: 27992409; PMID: 27272384; PMID: 27347757; PMID: 30892626; and PMID: 30704988), mouse embryos (e.g., PMID: 27272385; PMID: 28040780; PMID: 27272387; PMID: 31482512; and PMID: 31558757), zebrafish (e.g., PMID: 29222508; and PMID: 30892626), and plants (e.g., PMID: 30950179; PMID: 31965382; and PMID: 31055869).

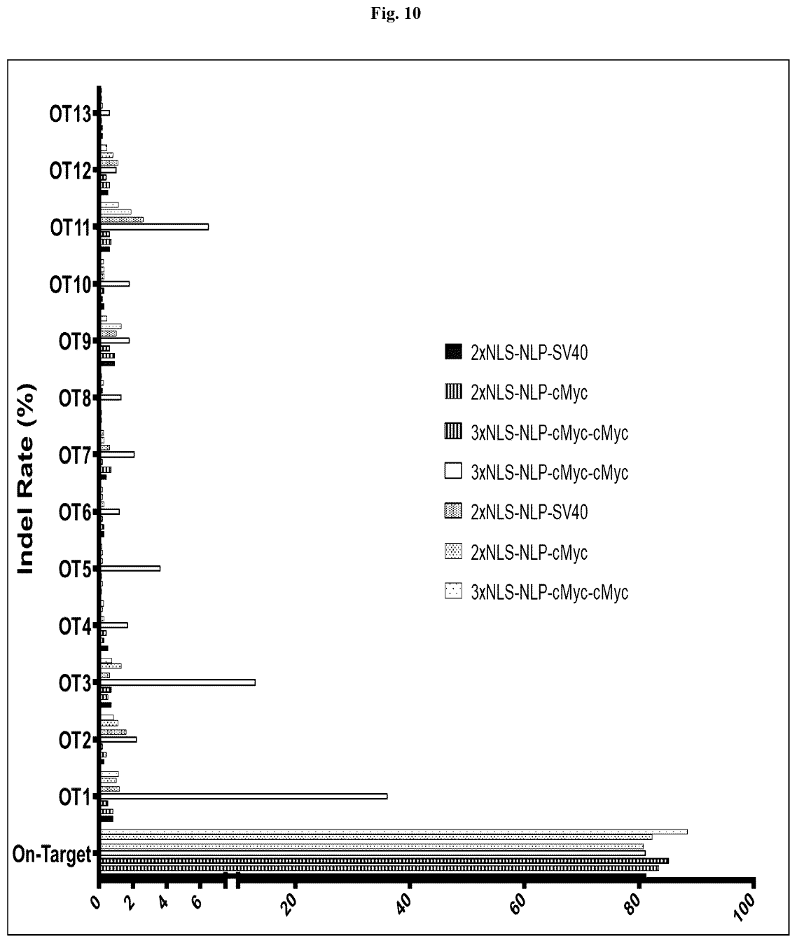

However, evaluations have shown that wild-type Cas12a nucleases have less robust activities than Cas9 in human cells (e.g., PMID: 30892626; PMID: 30892626; and PMID: 30892626). Therefore, many efforts have been made to increase overall activity and to expand the range of targetable sequences (e.g., PMID: 30892626; PMID: 28581492; PMID: 30742127; PMID: 30717767; and PMID: 32107556). Notably, enAspCas12a is a recently described Cas12a nuclease that is able to efficiently utilize a number PAM sequences (for example, TTYN, VTTV, TRTV, and others; where N=A/C/T/G, Y=C/T, and R=A/G), which provides an expanded targeting range relative to AspCas12a and LbaCas12a (PMID: 30742127). Additionally, enAspCas12a has demonstrated improved activity over AspCas12a and LbaCas12a at canonical-TTTV PAMs. However, enAspCas12a is more promiscuous, mutagenizing a higher number of off-target sites (PMID: 30742127) (e.g. see FIG. 10).

### II. Nuclear Localization Signal Proteins (NLS)

Classical NLSs can be further classified as either monopartite or bipartite. It is believed this difference is that two basic amino acid clusters in bipartite NLSs are separated by a relatively short spacer sequence (hence bipartite-2 parts), while monopartite NLSs are not separated. For example, the SV40 Large T-antigen (SV40) NLS having the sequence PKKKRKV (SEQ ID NO: 1) is a monopartite NLS (PMID 6096007). On the other hand, the nucleoplasmin (NLP) NLS having the sequence KR[PAATKKAGQA]KKKK (SEQ ID NO: 2) is an example of a bipartite NLS, where the two clusters of basic amino acids, separated by a spacer of about 10 amino acids (PMID 3417784). Similarly, the bipartite SV40 (BPSV40) NLS having the sequence KR[TADGSEFESP]KKKRKVE (SEQ ID NO: 3) is another example of a bipartite NLS, where the two clusters of basic amino acids, separated by a spacer of about 10 amino acids (PMID 19413990). The role of neutral and acidic amino acids was shown to contribute to the efficiency of the NLS (PMID 8805337). Nuclear localization efficiencies of eGFP fused NLSs were compared between SV40 large T-antigen, nucleoplasmin (AVKRPAATKKAGQAKKKKLD) (SEQ ID NO: 4), EGL-13 (MSRRRKANPTKLSENAKKLAKEVEN) (SEQ ID NO: 5), c-Myc (PAAKRVKLD) (SEQ ID NO: 6) and TUS-protein (KLKIKRPVK) (SEQ ID NO: 7) through rapid intracellular protein delivery, where significantly higher nuclear localization efficiency of c-Myc NLS was found as compared to that of SV40 NLS (PMID 26011555).

Nuclear localization signal (NLS) protein sequences are believed to play a role in the nuclear import of proteins. Previously, improvements in SpyCas9 gene editing in quiescent primary cells was investigated by optimizing the sequence composition and the number of NLS sequences. It was found that SpyCas9 bearing three NLSs (1 N-terminal and 2 C-terminal; 3×NLS-cMyc-SV40-NLP SpyCas9), and Cas12a bearing two C-terminal NLSs (2×NLS-SV40-NLP Cas12a) substantially improved editing activity in primary hematopoietic stem cells, transformed cell lines, and zebrafish likely through increased nuclear uptake of the protein (PMID: 30911135; and PMID: 30892626). While these improvements to Cas12a nuclease increased their activity in both transformed cell lines and zebrafish, the Cas12a with two NLS sequences (2×NLS-SV40-NLP) did not achieve the same level of targeted mutagenesis as 3×NLS-cMyc-SV40-NLP SpyCas9 in CD34+ hematopoietic stem and progenitor cells (HSPCs) at therapeutically relevant targets (PMID: 30704988).

These observations prompted further investigations to improve Cas12a editing activity in quiescent primary cells with a focus on examining the impact of the number and composition of NLS sequences on Cas12a ribonucleoprotein (RNP) editing rates. To improve the nuclear import of Cas12a, the SV40 T antigen NLS was substituted with the c-Myc NLS, and a third NLS was added to either N- or C-terminal of Cas12a. As shown in the data herein, these modifications to the NLS framework of Cas12a provided surprising, superior and unpredictable improved platforms for therapeutic genome editing.

### III. CRISPR-Cas/NLS Fusion Proteins

With Streptococcus pyogenes Cas9 (SpyCas9) it was found that improved nuclear localization signal (NLS) sequence composition and number can increase their activity when delivered to quiescent primary cells. Two NLS sequences on the C-terminus of Cas12a (2×NLS-SV40-NLP) was previously reported to result in increased nuclear import and editing efficiency in mammalian cells and zebrafish.

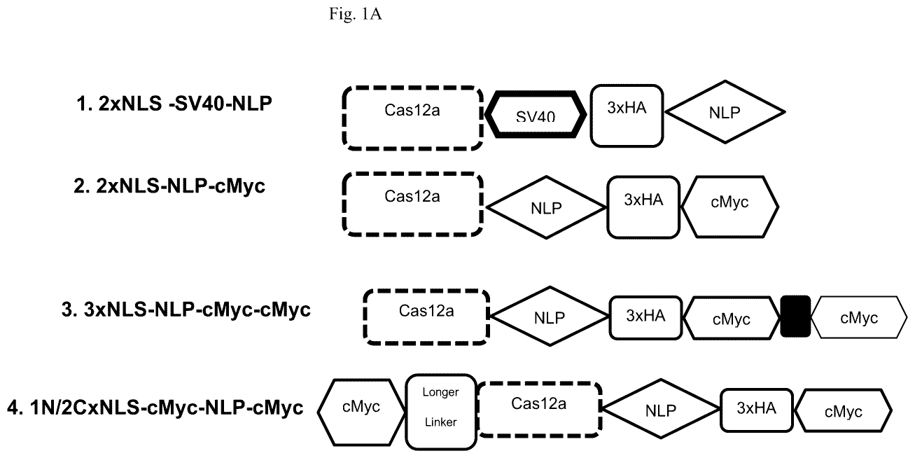

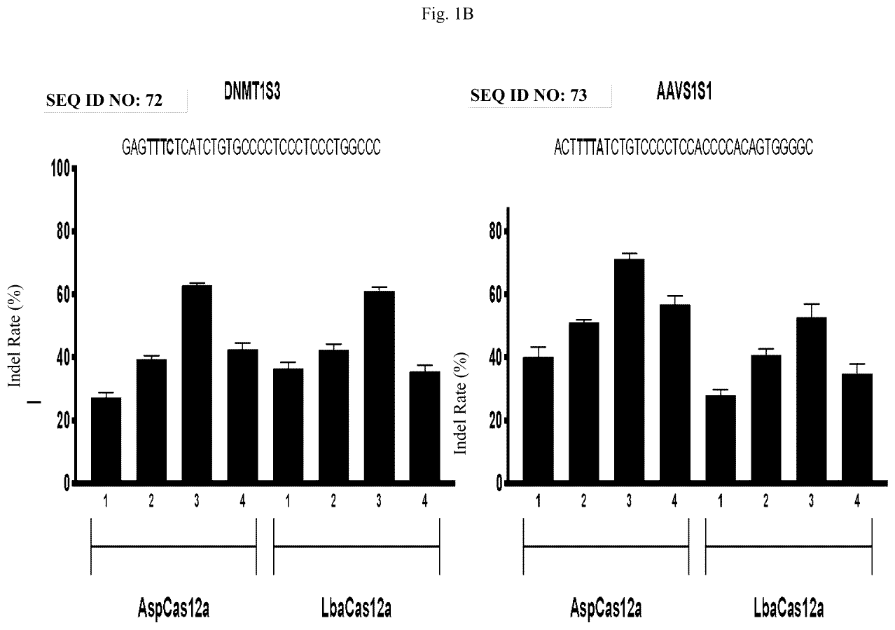

In one embodiment, the present invention contemplates compositions and method to improve Cas12a system activity with NLS modifications. In one embodiment, an improvement to a Cas12a\NLS architecture results in highly-efficient targeted mutagenesis in mammalian and primary cells. Although it is not necessary to understand the mechanism of an invention, it is believed that an improved Cas12a/NLS architecture may be achieved by substitution of a low efficiency SV40 T antigen NLS sequence with a high efficiency c-Myc NLS sequence. It was further believed that by increasing in the number of NLS sequences fused to the Cas12a protein from two to three would further improve nuclear entry and result in a more robust Cas protein platform. FIG. 1A.

After construction and purification of several AspCas12a and LbaCas12a NLS variants, the activities of these proteins were characterized when delivered into human cells as ribonucleoprotein (RNP) complexes by electroporation. The data showed that when three C-terminal NLS sequences were fused to the Cas12a protein a 1.25-to-3 fold increase in editing efficiency was produced in HEK293T, Jurkat, and K562 cells at subsaturating concentrations. FIG. 1B. In one embodiment, the present invention contemplates a Cas12a/c-Myc NLS fusion protein for improved therapeutic genome editing as compared to conventional Cas12a/NLS gene editing platforms.

### A. Cas12a Nuclear Localization Signal Sequence Architecture

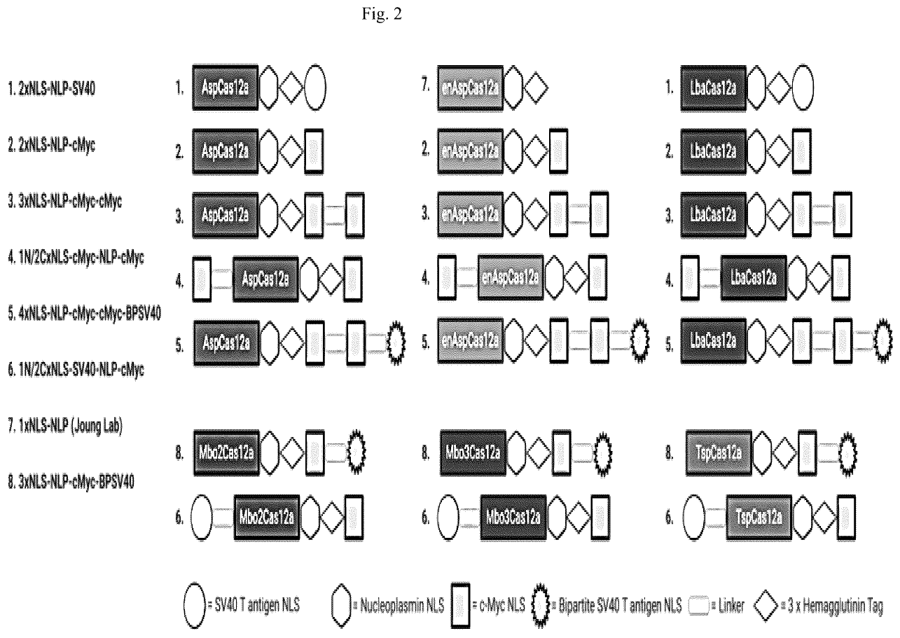

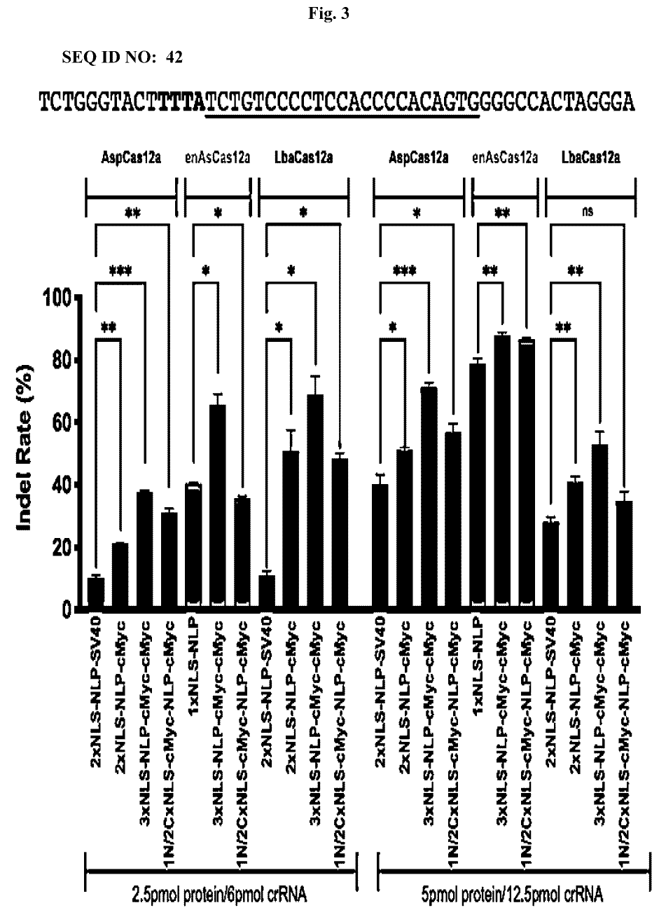

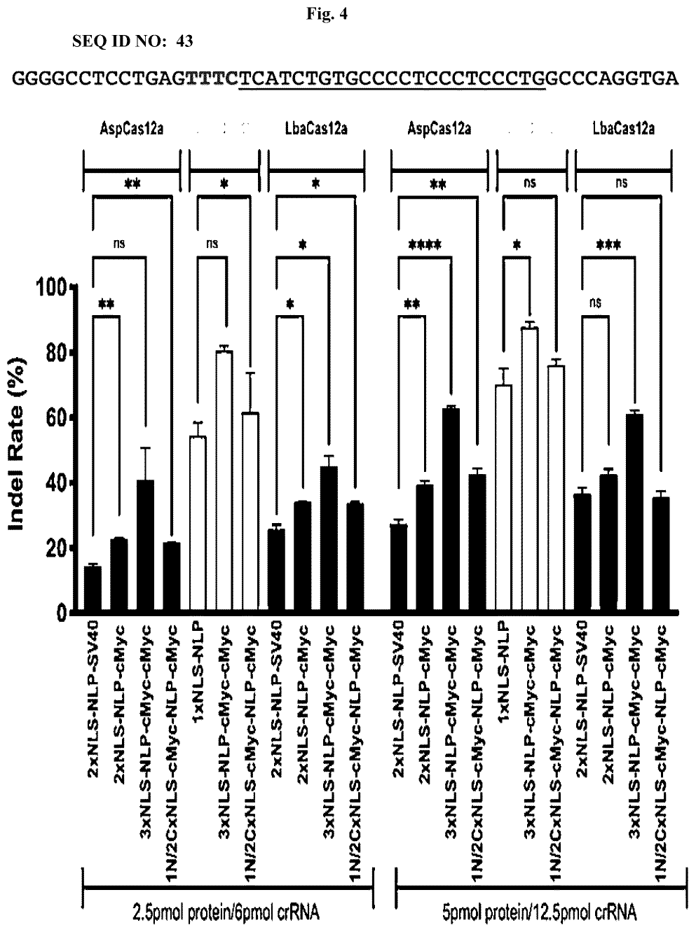

In one embodiment, the present invention contemplates a Cas12a nuclease for targeted mutagenesis (e.g., gene editing) in mammalian and quiescent primary cells. In one embodiment, the Cas12a nuclease includes, but is not limited to, AspCas12a, enAspCas12a, and LbaCas12a. In one embodiment, the Cas12a nuclease comprises a c-Myc NLS sequence (PMID 26011555). Although it is not necessary to understand the mechanism of an invention, it is believed that nuclear localization efficiency is not limited to NLS type, but also NLS position within the CRISPR-Cas12a fusion protein sequence. For example, the data presented herein shows that the N-terminal c-myc NLS tag in the context of the 3×NLS Cas12a constructs do not work as well as the construct where all of the NLSs are present as C-terminal tags. For example, in construct versions 4 and 5, the c-Myc NLSs differ in position between the N or C terminus. FIG. 2. The data show that the 1N/2C constructs (comprising an additional N-terminal C-myc NLS) is always less effective than the 3×NLS (comprising an additional C-terminal C-myc NLS). FIGS. 3 and 4. Similar comparative data is seen in the AspCas12a and LbaCas12a constructs as well.

In one embodiment, the Cas12a nuclease comprises a plurality of c-Myc NLS sequences. In one embodiment, the Cas12a nuclease comprises at least one c-Myc NLS sequence. In one embodiment, the Cas12a nuclease comprises two c-Myc NLS sequences. In one embodiment, the Cas12a nuclease comprises three c-Myc NLS sequences. Although it is not necessary to understand the mechanism of an invention, it is believed that an increased number of NLS sequences facilitates more effective nuclear entry and results in a more robust genome editing platform.

In one embodiment, the present invention contemplates a plurality of Cas12a fusion proteins each with different NLS sequence configurations. FIG. 2. Following construction, these fusion proteins were purified from an E. coli overexpression system by Ni-NTA resin followed by cation exchange chromatography for comparative editing analysis.

The influence of the different NLS frameworks was examined using AspCas12a, enAspCas12a, and LbaCas12a at two previously defined active target sites in human cells (AAVS1S1; PMID: 30704988) and EMX1S1; PMID: 26422227; and PMID: 30892626). These ribonucleoproteins (RNPs) were delivered by electroporation to HEK293T cells at subsaturating concentrations (2.5 pmoles Cas12a protein:crRNA complex and 5 pmoles Cas12a protein:crRNA complex, respectively).

At both genomic target sites, it was observed that the substitution of a SV40 T-antigen NLS sequence for a c-Myc NLS sequence significantly increased the activity of the Cas12a nucleases. Further improvement in activity was achieved by the addition of a third NLS (c-Myc NLS) to the C-terminus of Cas12a irrespective of the Cas12a ortholog. FIGS. 3 and 4. Together, these results suggest that any Cas12a ortholog bearing a plurality of C-terminal NLS sequences (e.g., 3×NLS-NLP-cMyc-cMyc Cas12a) can significantly increase lesion frequencies at their respective nucleic acid target sites.

To determine the validity of these observations, a representative group of Cas12a variants including 2×NLS-SV40-NLP AspCas12a, 2×NLS-NLP-cMyc AspCas12a, 3×NLS-NLP-cMyc-cMyc AspCas12a, 3×NLS-NLP-cMyc-cMyc enAspCas12a, 2×NLS-SV40-NLP LbaCas12a, 2×NLS-NLP-cMyc LbaCas12a, and 3×NLS-NLP-cMyc-cMyc LbaCas12a were purified with the inclusion of a third size-exclusion chromatography step. See, Tables 1-3.

TABLE 1
Direct repeat designs for
Asp/enAsp/Lba/Mbo2/Mbo3/TsCas12a:
Italics = Removed by after pre-crRNA maturation
Underline = Direct repeat region
Black = Stem loop
Bold = Spacer sequence

| | SEQ | |
|---|---|---|
| Cas12a | ID | |
| Nucleases | NOS: | Direct Repeat Design |
| AspCas12a/ | 8 | GUCAAAAGACCUUUUUAAUUUCU |
| enAspCas12a | | ACUCUUGUAGAU + 23 nt Spacer |
| LbaCas12a | 9 | GUUUCAAAGAUUAAAUAAUUUCU |
| | | ACUAAGUGUAGAU + 23 nt Spacer |
| Mbo2Cas12a/ | 10 | GUCUAACUACCUUUUAAUUUCUA |
| Mbo3Cas12a | | CUGUUUGUAGAU + 23 nt Spacer |
| TspCas12a | 11 | CUCUAGCAGGCCUGGCAAAUUUC |
| | | UACUGUUGUAGAU + 23 nt Spacer |

TABLE 2
Synthetic crRNAs for Asp/enAsp/LbaCas12a:
Underline = Direct repeat region
Bold = Spacer sequence

| | | SEQ | |
|---|---|---|---|
| Target | | ID | |
| Site | Nucleases | NOS: | Sequence |
| AAVS1S1 | AspCas12a/ | 12 | UAAUUUCUACUCUUGUAGAU |
| | enAspCas12a | | UCUGUCCCCUCCACCCCACA |
| | | | GUG |
| AAVS1S1 | LbaCas12 | 13 | UAAUUUCUACUAAGUGUAGAU |
| | | | UCUGUCCCCUCCACCCCACAG |
| | | | UG |
| EMX1S1 | AspCas12a/ | 14 | UAAUUUCUACUCUUGUAGAU |
| | enAspCas12a | | UCAUCUGUGCCCCUCCCUCCC |
| | | | UG |
| EMX1S1 | LbaCas12 | 15 | UAAUUUCUACUAAGUGUAGAU |
| | | | UCAUCUGUGCCCCUCCCUCCC |
| | | | UG |
| DNMT1S | AspCas12a/ | 16 | UAAUUUCUACUCUUGUAGAU |
| 3 | enAspCas12a | | CUGAUGGUCCAUGUCUGUUA |
| | | | CUC |
| DNMT1S | LbaCas12 | 17 | UAAUUUCUACUAAGUGUAGAU |
| 3 | | | CUGAUGGUCCAUGUCUGUUAC |
| | | | UC |
| BCL11A | AspCas12a/ | 18 | UAAUUUCUACUCUUGUAGAU |
| TS1 | enAspCas12a | | CUGGAGCCUGUGAUAAAGCA |
| | | | ACU |
| BCL11A | AspCas12a/ | 19 | UAAUUUCUACUCUUGUAGAU |
| TS2 | enAspCas12a | | ATCACAGGCTCCAGGAAGGG |
| | | | TTTG |

TABLE 3
T7 in vitro transcribed crRNAs for
Mbo2/Mbo3/TspCas12a:
Italics = Removed by post crRNA maturation
Underline = Direct repeat region
Black = Stem loop
Bold = Spacer sequence

| | | SEQ | |
|---|---|---|---|
| Target | | ID | |
| Site | Nucleases | NOS: | Sequence |
| HEXA | TspCas12a | 20 | CUCUAGCAGGCCUGGCAAAUUUC |
| | | | UACUGUUGUAGAUCAGUCAGG |
| | | | GCCAUAGGAUAGAUAU |
| HEXA | Mbo2Cas12a/ | 21 | GUCUAACUACCUUUUAAUUUCU |
| | Mbo3Cas12a | | ACUGUUUGUAGAUCAGUCAGG |
| | | | GCCAUAGGAUAGAUAU |
| EMX1 | TspCas12a | 22 | CUCUAGCAGGCCUGGCAAAUUUC |
| | TTTC TS1 | | UACUGUUGUAGAUUCAUCUGUG |
| | | | CCCCUCCCUCCCUG |
| EMX1 | TspCas12a | 23 | CUCUAGCAGGCCUGGCAAAUUUC |
| | CTTC TS2 | | UACUGUUGUAGAUCCAUCAGGC |
| | | | UCUCAGCUCAGCCU |
| EMX1 | TspCas12a | 24 | CUCUAGCAGGCCUGGCAAAUUUC |
| | GTTG TS3 | | UACUGUUGUAGAUAGGCCCCA |
| | | | GUGGCUGCUCUGGGG |
| EMX1 | Mbo2Cas12a/ | 25 | GUCUAACUACCUUUUAAUUUCU |
| | Mbo3Cas12a | | ACUGUUUGUAGAUUCAUCUGUG |
| | TTTC TS1 | | CCCCUCCCUCCCUG |
| EMX1 | Mbo2Cas12a/ | 26 | GUCUAACUACCUUUUAAUUUCU |
| | Mbo3Cas12a | | ACUGUUUGUAGAUCCAUCAGGC |
| | CTTC TS2 | | UCUCAGCUCAGCCU |
| EMX1 | Mbo2Cas12a/ | 27 | GUCUAACUACCUUUUAAUUUCU |
| | Mbo3Cas12a | | ACUGUUUGUAGAUAGGCCCCA |
| | GTTG TS3 | | GUGGCUGCUCUGGGG |

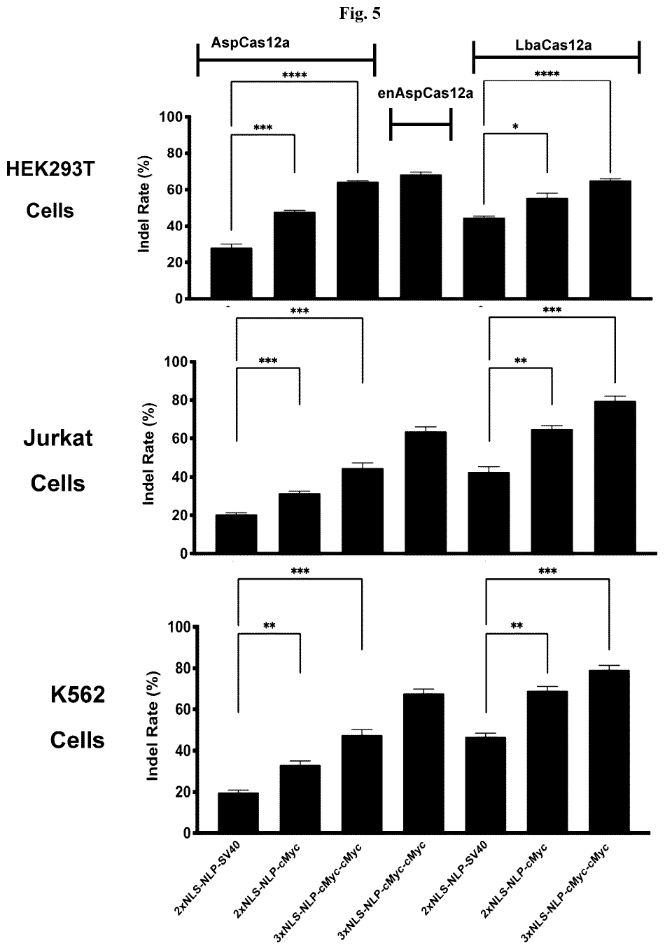

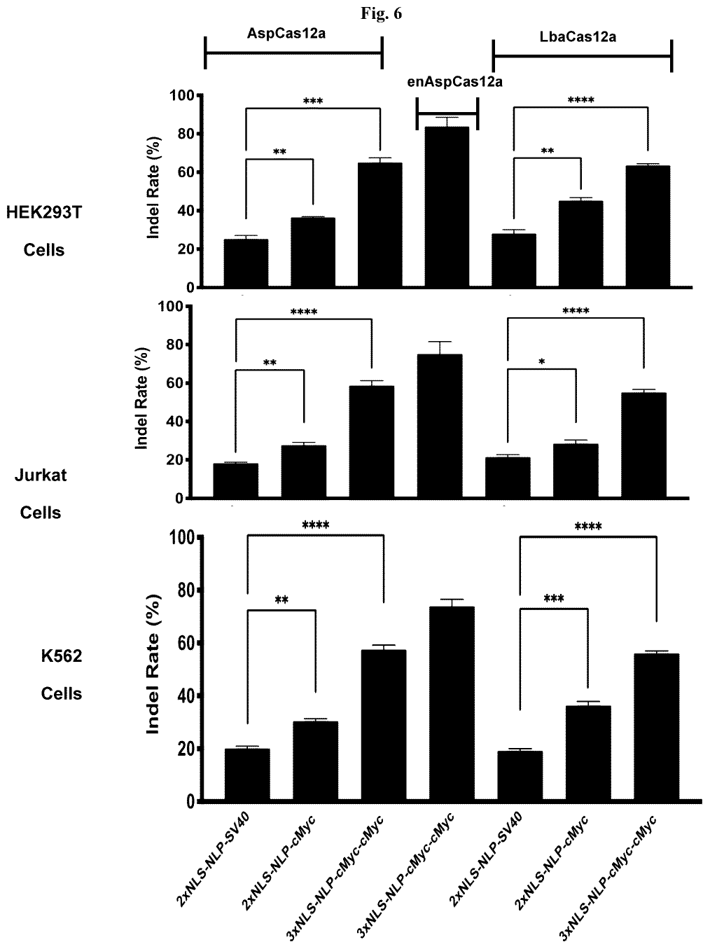

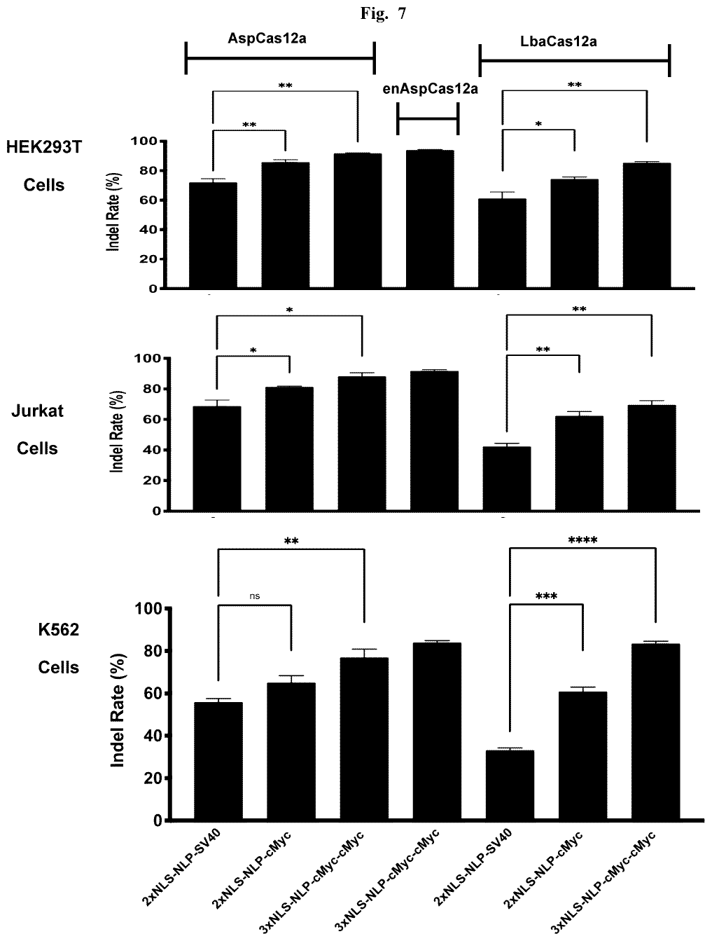

Gene editing of these proteins was assessed as Cas12a RNPs at three HEK293T, K562, and Jurkat cell genomic target sites (e.g., AASVS1, EMX1S1, and DNMT1S3) delivered by electroporation. Consistent with the above data, substitution of a SV40 T antigen NLS sequence for a c-Myc NLS sequence and the fusion of three C-terminal NLSs resulted in a 1.25-to-3 fold increase in activity across all cell lines. FIGS. 5, 6, and 7.

### B. Cas12a Specificity

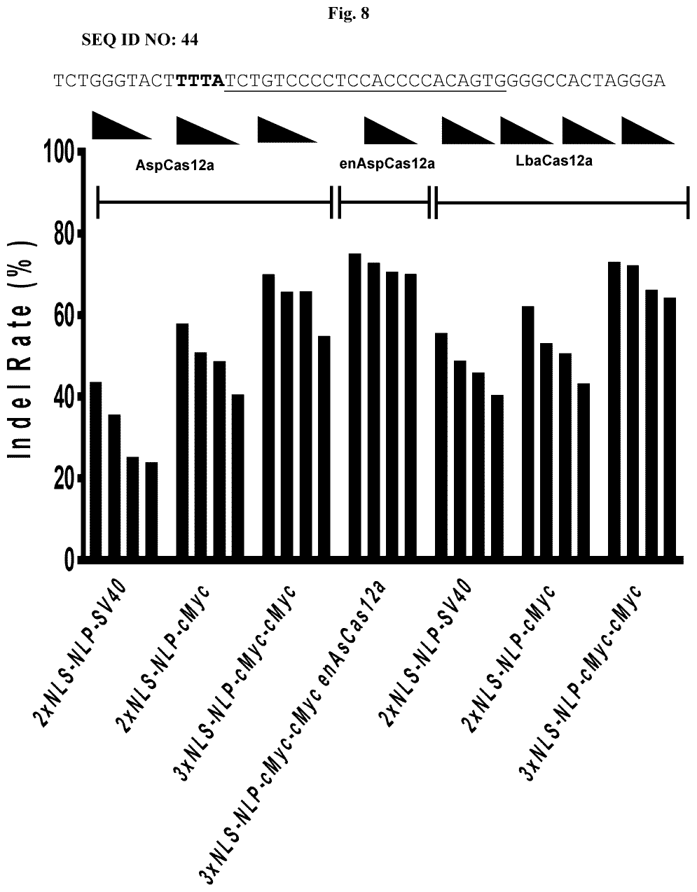

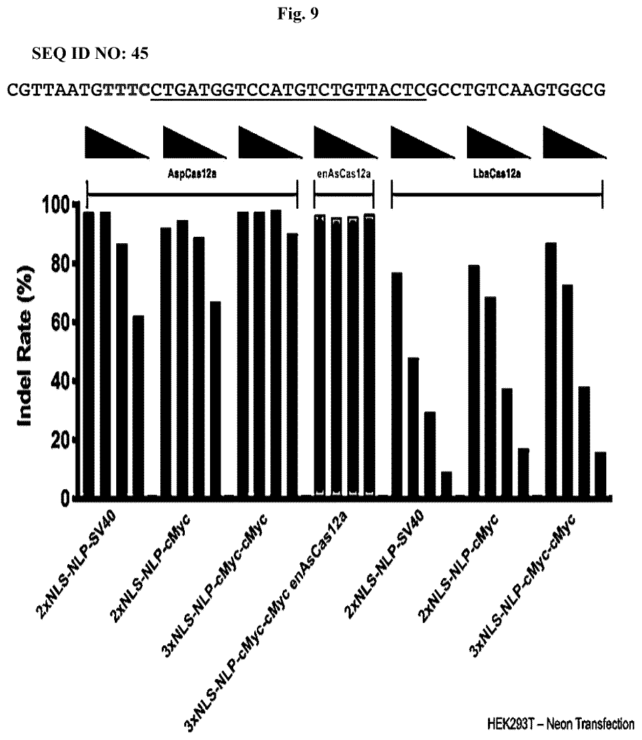

To compare the specificity of the Cas12a proteins with different NLS frameworks, titration experiments were carried out in HEK293T cells where all nucleases would have similar near saturating on-target editing rates. FIGS. 8 and 9. For an off-target analysis, a crRNA targeting DNMT1S3 was used, which has a well-characterized off-target profile by both targeted and genome-wide deep sequencing approaches (PMID: 27272384; PMID: 27347757; and PMID: 30892626); FIG. 10. It was observed that the most active variants (e.g., 3×NLS-NLP-cMyc-cMyc AspCas12a and 3×NLS-NLP-cMyc-cMyc enAspCas12a) required delivery of lower amounts of Cas12a protein-crRNA complex to achieve similar on-target nuclease activities. FIGS. 8 and 9.

HEK293T cells were then nucleofected with optimal concentrations of each RNP to achieve ˜80% editing and performed targeted deep sequencing analyses of PCR products spanning the target site and 13 potential off-target sites for the DNMT1S3 crRNA to measure the rate of indels. Comparison of previously described 2×NLS-SV40-NLP Cas12a with the presently disclosed Cas12a NLS variants (e.g., 2×NLS-NLP-cMyc Cas12a and 3×NLS-NLP-cMyc-cMyc Cas12a) demonstrates that neither the substitution of the SV40 T-antigen NLS for c-Myc NLS nor the addition of a third NLS sequence significantly increases the gene editing rates at any of the previously validated off-target sites. FIG. 10. Furthermore, enAspCas12a was the most promiscuous nuclease at all 13 off-target sites, consistent with previous examinations of the specificity of enAspCas12a (PMID: 30742127). FIG. 10. Together, these data show that the modifications to NLS architecture increase the on-target activity of Cas12a but do not substantially alter their specificity (e.g., by not concomitantly increasing off-target activity).

### C. Genetic Regulatory Element Targeting

In one embodiment, the present invention contemplates a Cas12a/NLS nuclease platform targeted at a therapeutically relevant genomic locus. For example, an enAspCas12a was tested for disrupt ion of a GATA1-binding motif regulatory element within the BCL11A erythroid-lineage-specific enhancer (+58 kb) element (PMID: 26375006; and PMID: 26322838). Disruption of the GATA1-binding motif regulatory element in CD34+ HSPCs silences BCL11A expression in the erythroid lineage and results in increased production of fetal γ-globin protein in differentiated red blood cells (PMID: 28344999; and PMID: 30911135). These observations suggest that ex vivo editing of the +58 kb element in CD34+ HSPCs in conjunction with autologous bone marrow transplantation is a potential treatment of beta-hemoglobinopathies (PMID: 30911135).

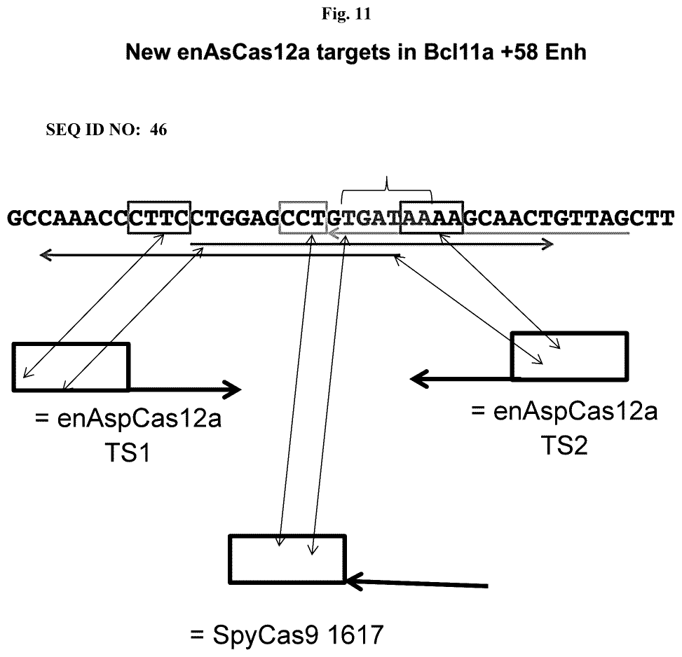

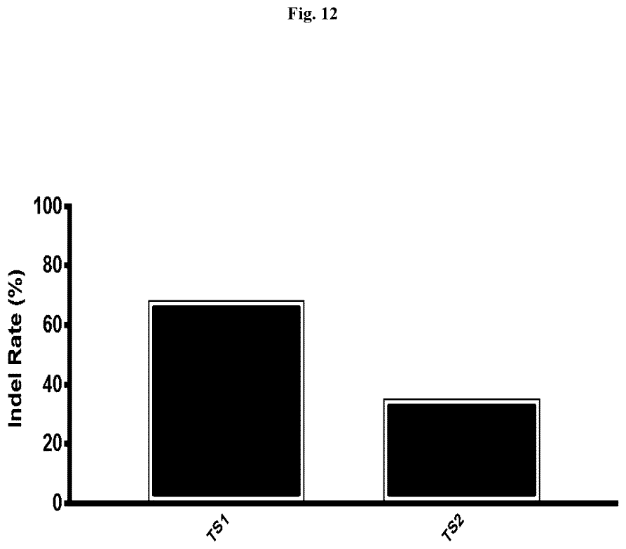

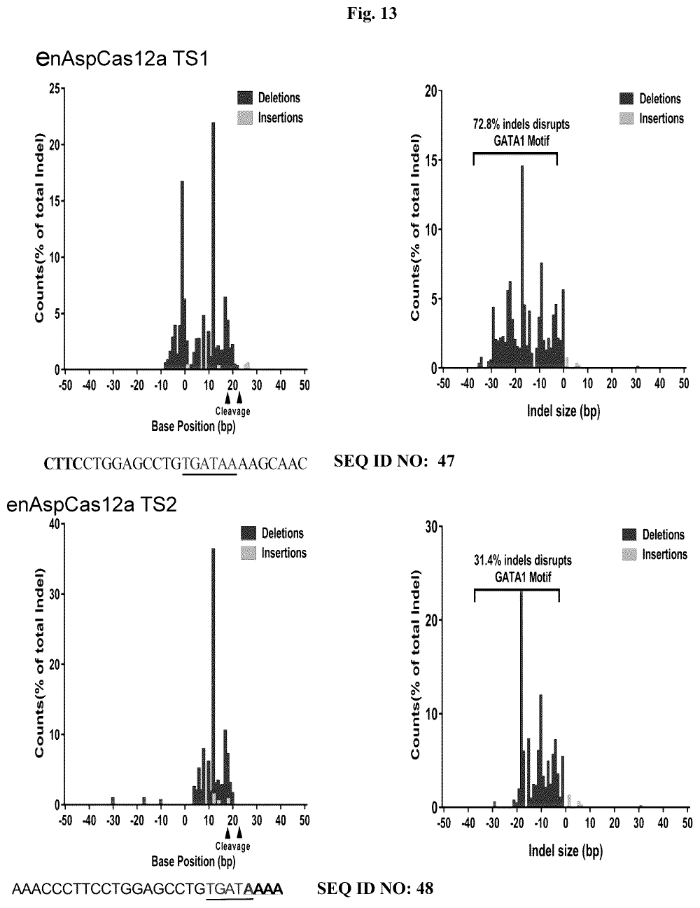

The data presented herein demonstrates the targeting of 3×NLS-NLP-cMyc-cMyc enAspCas12a to two different sites that overlap a GATA1-binding motif in HEK293T cells FIG. 11. It was found that both crRNAs were able to effectively mutagenize the regulatory element (TS1=˜70% and TS2=˜40%). However, between the two target sites, we observed that TS1 was more efficient in overall activity. FIG. 12. Furthermore, examination of the indel spectrum produced by each respective crRNAs, demonstrated that >70% of the indels produced by TS1 disrupted the GATA1-binding motif. FIG. 13. These data suggest that the presently disclosed Cas12a NLS variants are a useful platform for the disruption of functional sequence motifs in therapeutically relevant genomic loci.

### D. Protospacer Adjacent Motif Range Expansion

While AspCas12a and LbaCas12a are the most commonly utilized Cas12a orthologs, new Cas12a orthologs have been recently reported (PMID: 31723075; and PMID: 30717767). Most notably, Cas12a orthologs from Moraxella bovoculi AAX08_00205 (Mbo2Cas12a), Moraxella bovoculi AAX11_00205 (Mbo3Cas12a), and Thiomicrospira sp. Xs5 (TspCas12a) have been shown to induce indels in human cells (PMID: 31723075). While AspCas12a and LbaCas12a use a TTTV PAM sequence, Mbo2Cas12, Mbo3Cas12a, and TspCas12a recognize the less-restrictive NTTN PAM sequence in bacterial PAM analysis assays (PMID 31723075). Based on these characteristics, the presently disclosed NLS frameworks were investigated using these new Cas12a orthologs. FIG. 2.

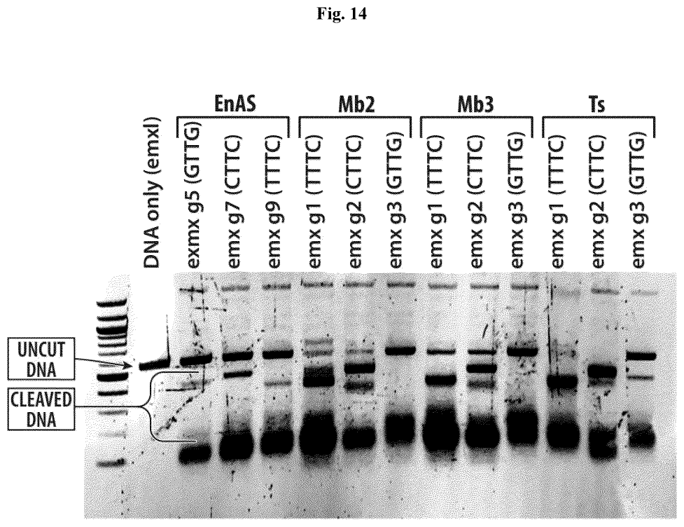

To test these recently reported ortholog Cas12a nucleases, 1N/2CxNLS-cMyc-NLP-cMyc fusion proteins of these Cas12a nucleases were purified. An in vitro cleavage assay was then performed using three different EMX1 target sites bearing three different PAM sequences (TTTC, CTTC, and GTTG) and their activity was compared to that of enAspCas12a. The data show that these ortholog Cas12a nucleases were active at varying levels at all three target sites. FIG. 14.

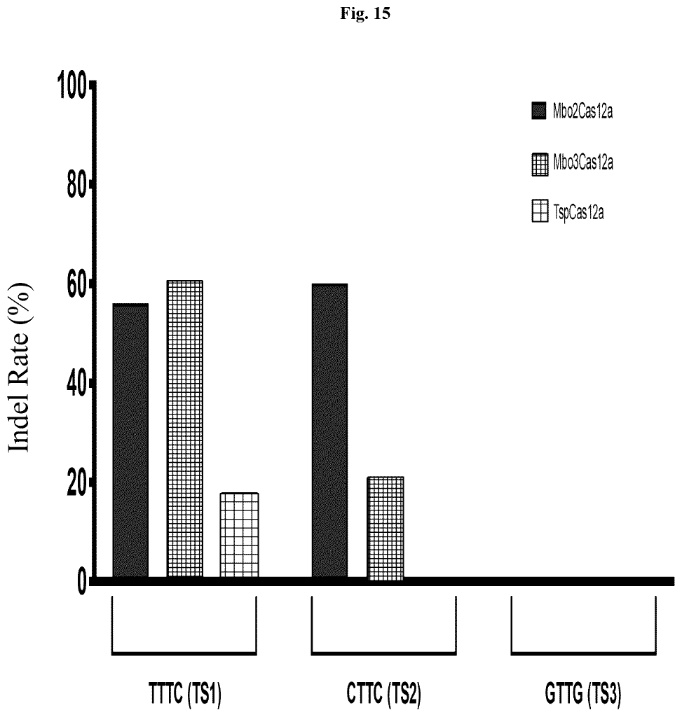

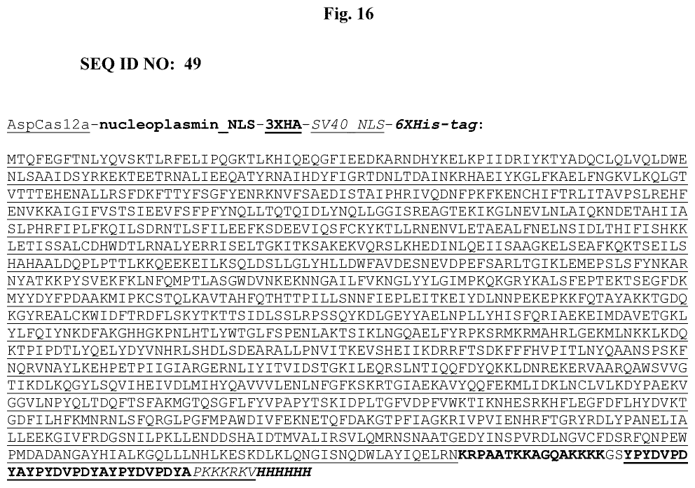

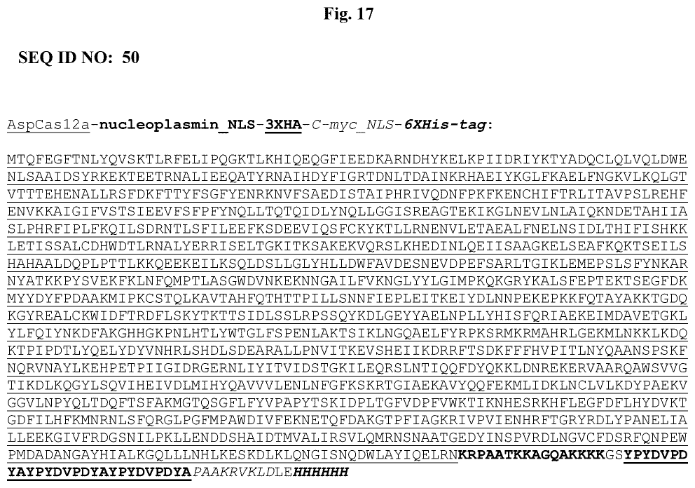

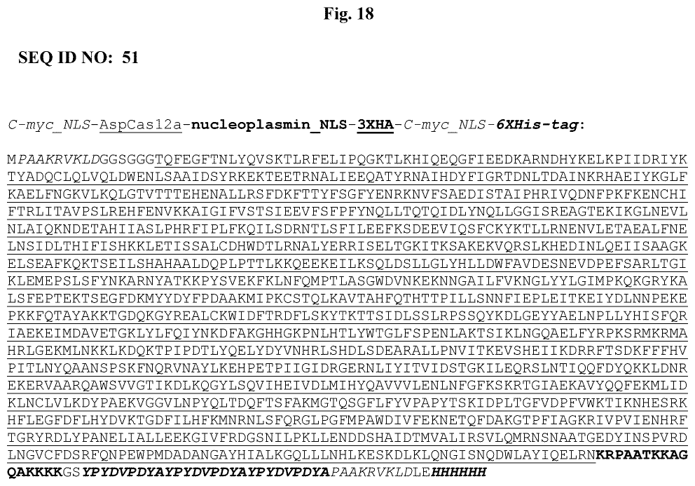

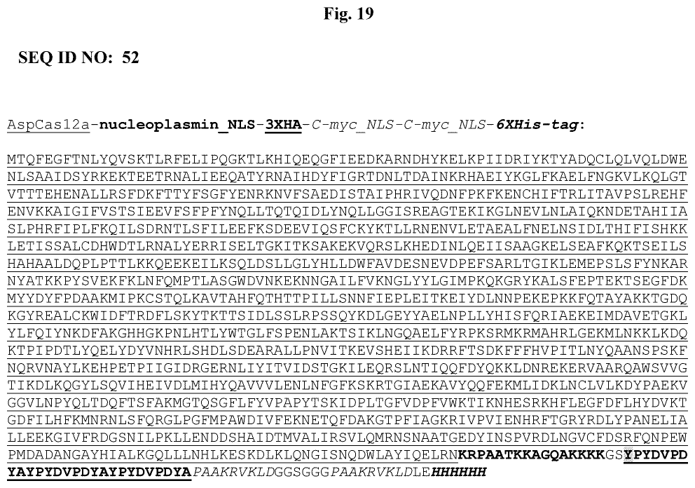

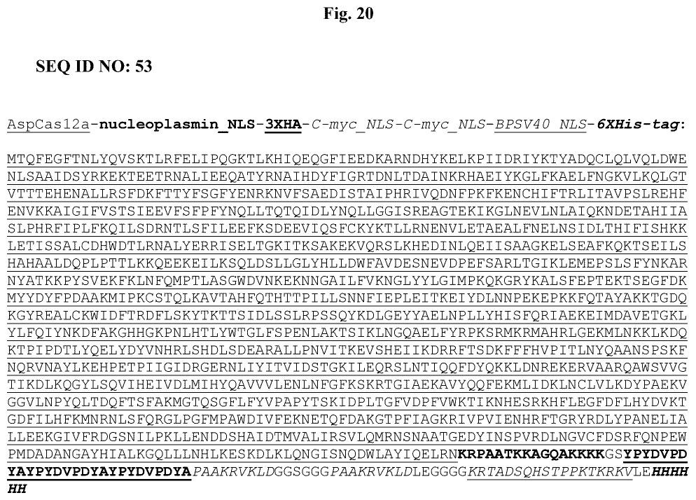

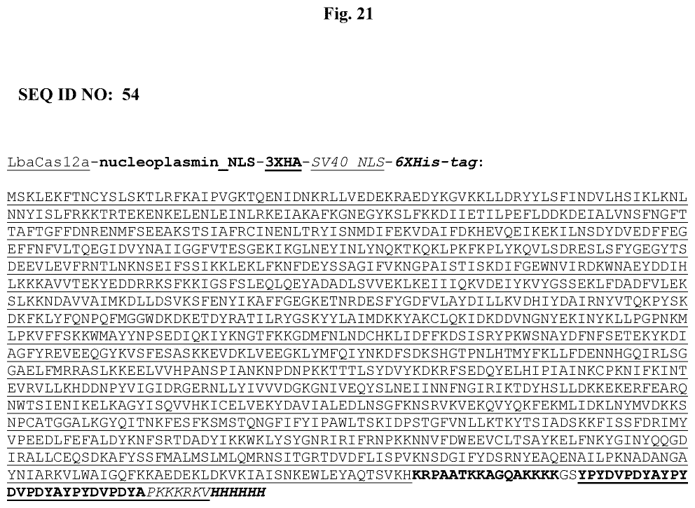

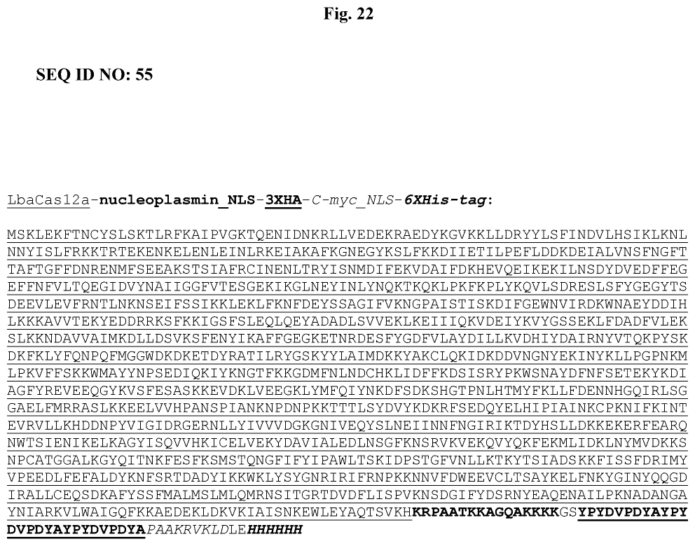

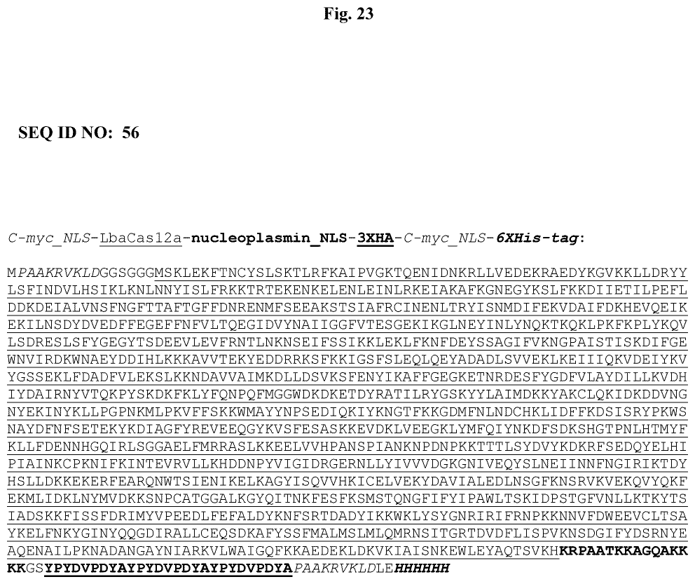

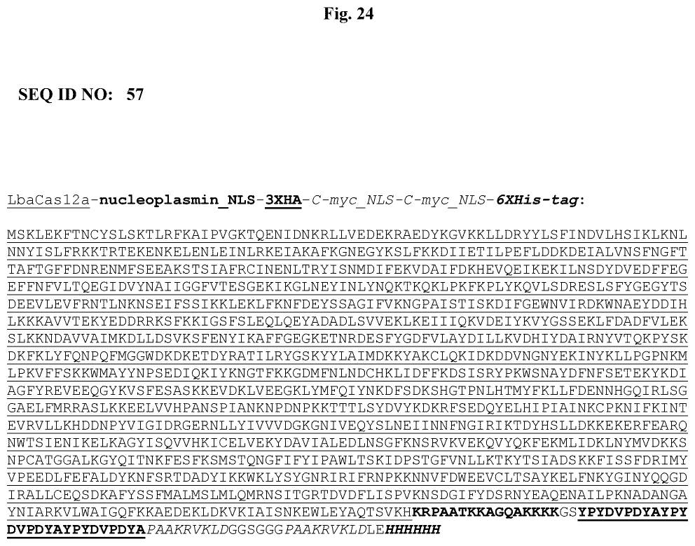

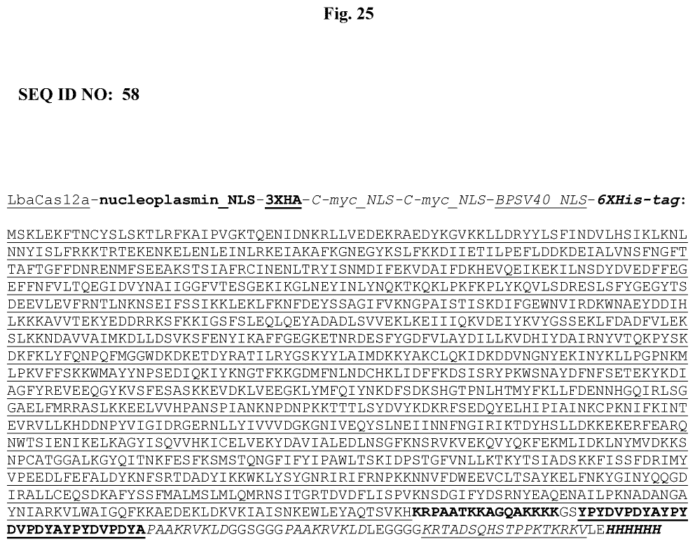

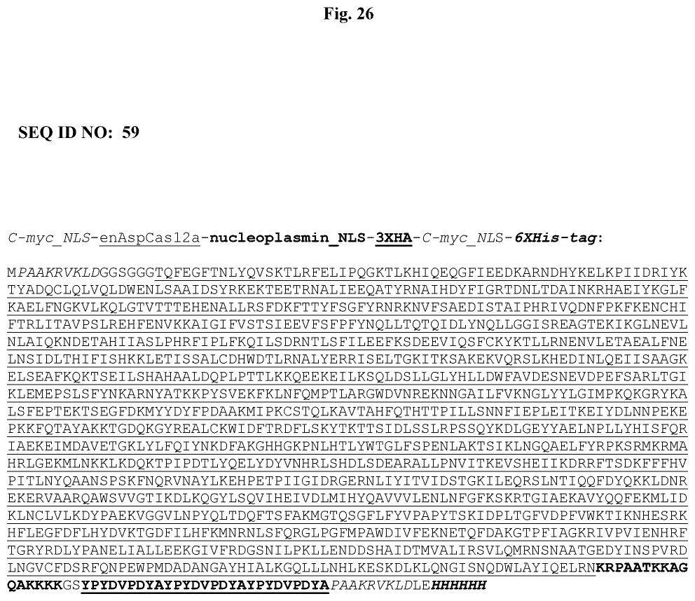

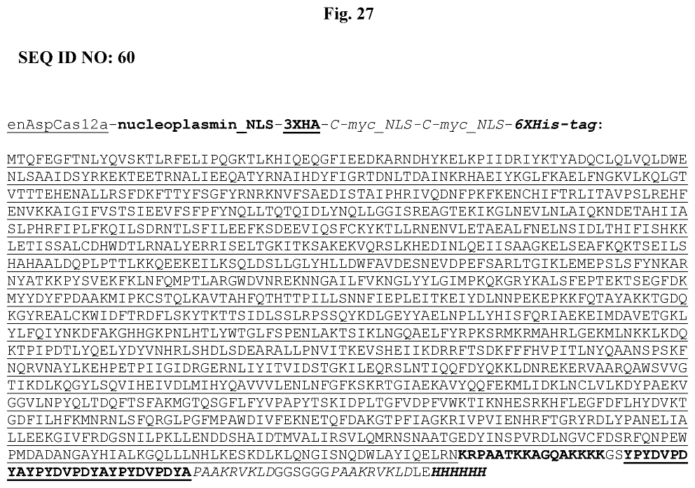

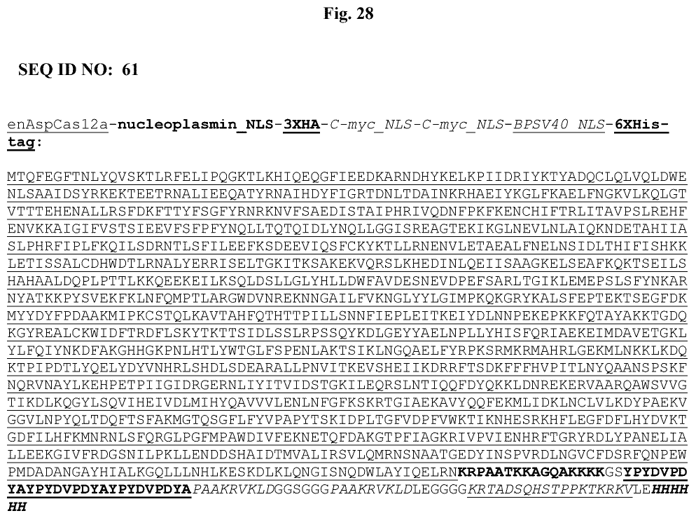

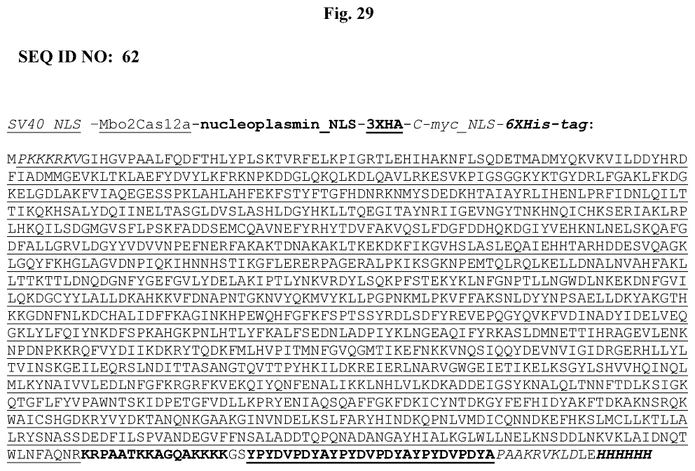

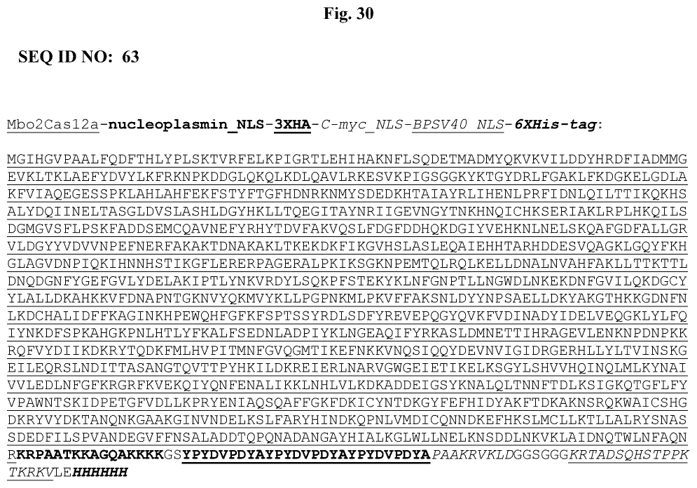

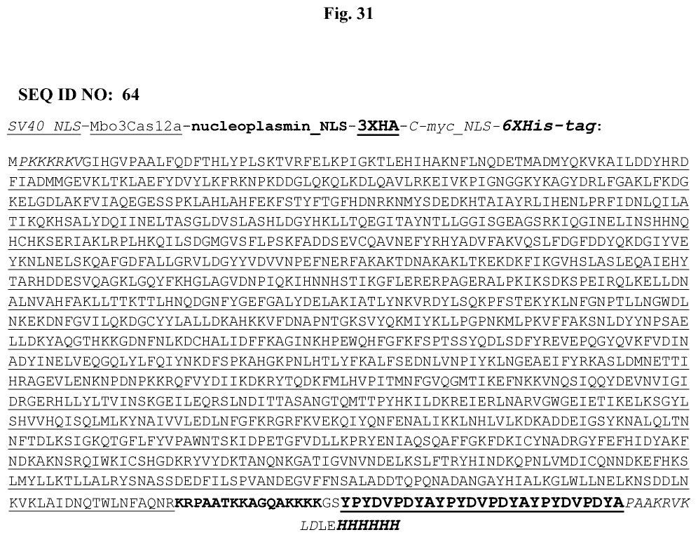

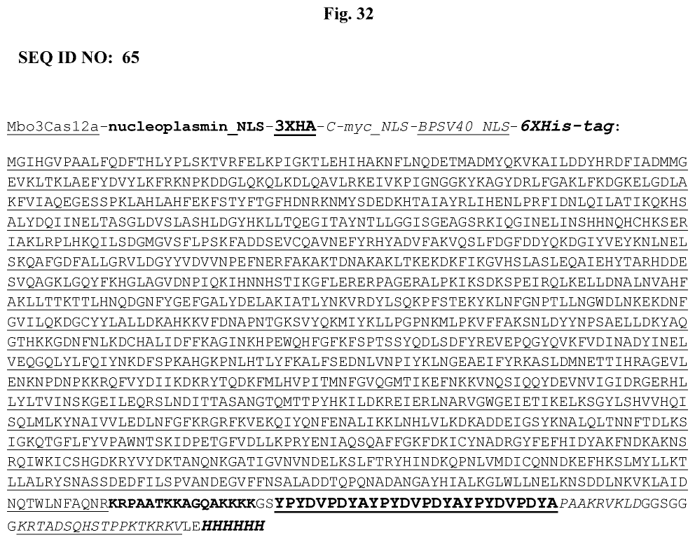

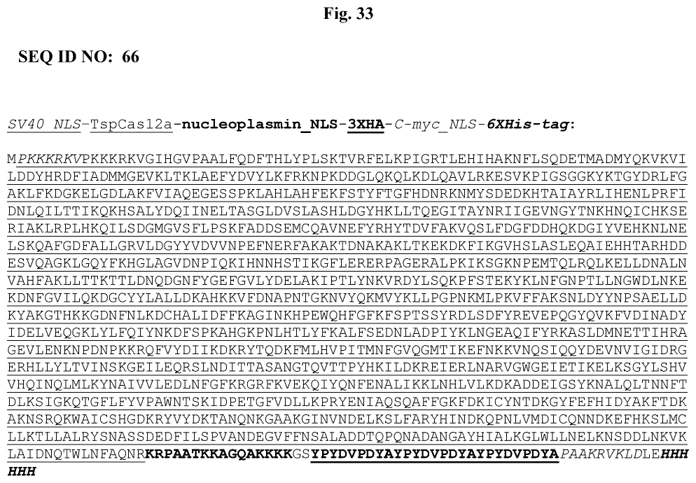

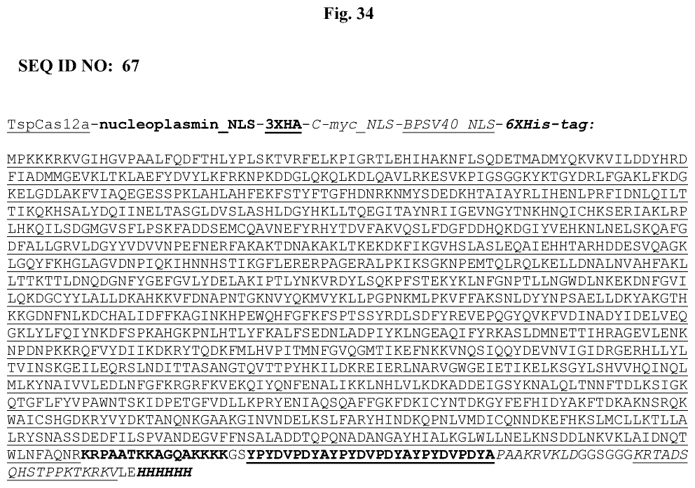

To confirm the activity of theses recently reported Cas12a nucleases in mammalian cell lines, HEK293T cells were nucleofected with Mbo2Cas12a, Mbo3Cas12a, and TspCas12a fused to the NLS variants disclosed herein that targeted EMX1 at the same three target sites utilized in the above in vitro cleavage assay. Unlike the in vitro cleavage experiment, the data shows that Mbo2Cas12a and Mbo3Cas12a were active at TTTC and CTTC PAM sequences, while TspCas12a was only active at the TTTC PAM target site. Furthermore, Mbo2Cas12a was substantially more active than Mbo3Cas12a at the CTTC PAM EMX1 TS2. FIG. 15. Notably, previous evaluation of Mbo3Cas12a at CTTC PAM containing target sites had yielded little to no editing activity (<10%) (PMID 31723075). Together, these data demonstrate that Mbo2Cas12a and Mbo3Cas12a are Cas12a orthologs that are active at non-TTTV PAM sequences when constructed as a fusion protein with the presently disclosed NLS frameworks.

Representative PAM sequences for specific target sites of the presently disclosed NLS variant Cas12 nucleases are presented in Table 4.

TABLE 4
Representative PAM sequences at
specific target sites.

| | SEQ | |
|---|---|---|
| | ID | Protospacer |
| Target Site Name | NOS: | Sequence |
| enAspCas12a IFNG Exon 2 | 28 | AAGATGACCAG |
| TS | | AGCATCCAAAAG |
| enAspCas12a CD96 TS | 29 | GTCTGAATGCC |
| | | CTCTGGATACAG |
| +55 Enh ATF4 motif Spy | 30 | CTCCAAGCATT |
| TS1 | | GCATCATCC |
| +55 Enh ATF4 motif Spy | 31 | TACCAGGATGA |
| TS2 | | TGCAATGCT |
| +55 Enh ATF4 motif | 32 | CCTTCCTGGTA |
| enAsp/Mb2 TS1 | | CCAGGATGATGC |
| +55 Enh ATF4 motif | 33 | CTGGTACCAGGA |
| enAsp/Mb2 TS2 | | TGATGCAATGC |
| +58 Enh 1617 Spy | 34 | CTAACAGTTGCT |
| | | TTTATCAC |
| enAspCas12a AAVS1S1 | 35 | TCTGTCCCCTCCA |
| | | CCCCACAGTG |

### IV. Cas12a Editing to Correct Tay-Sachs Disease

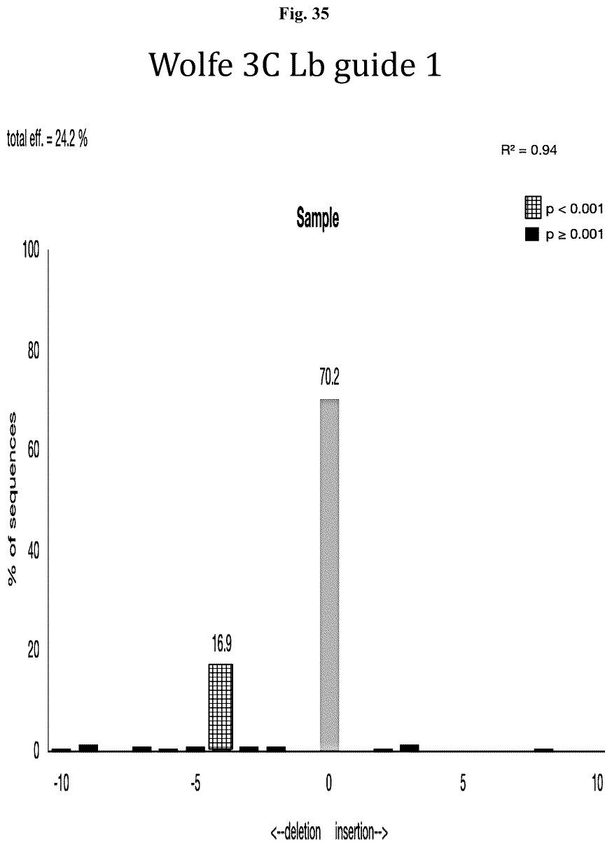

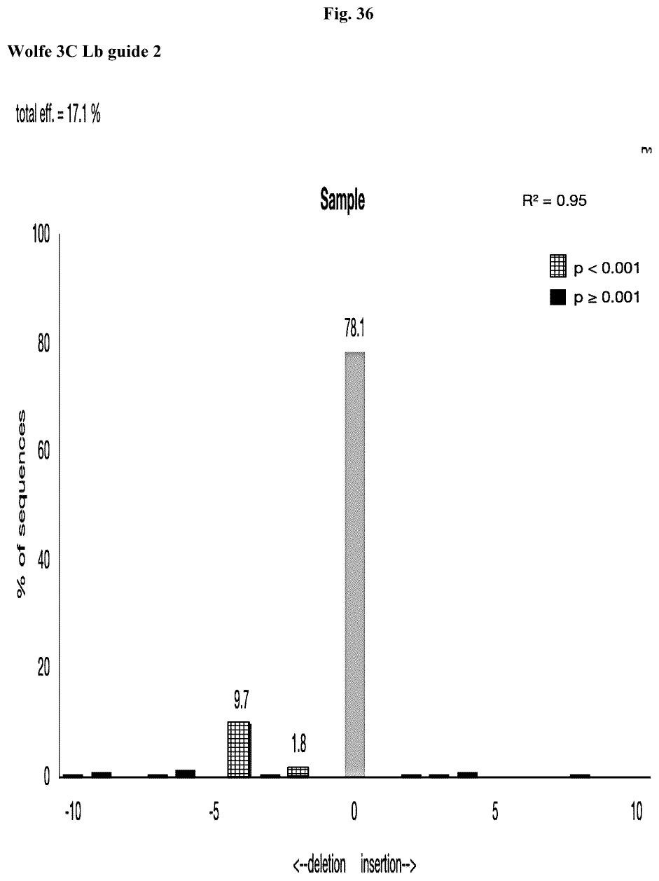

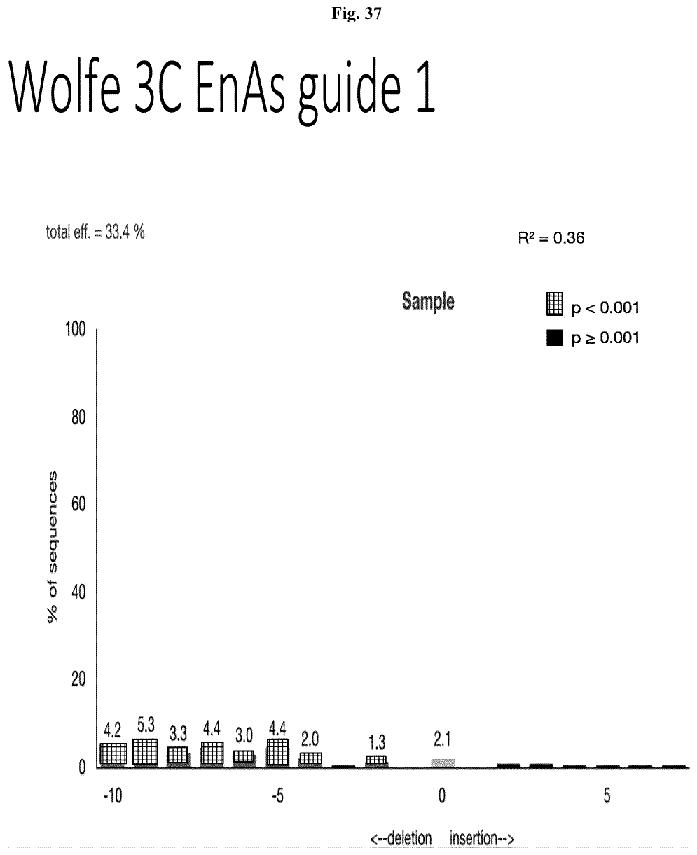

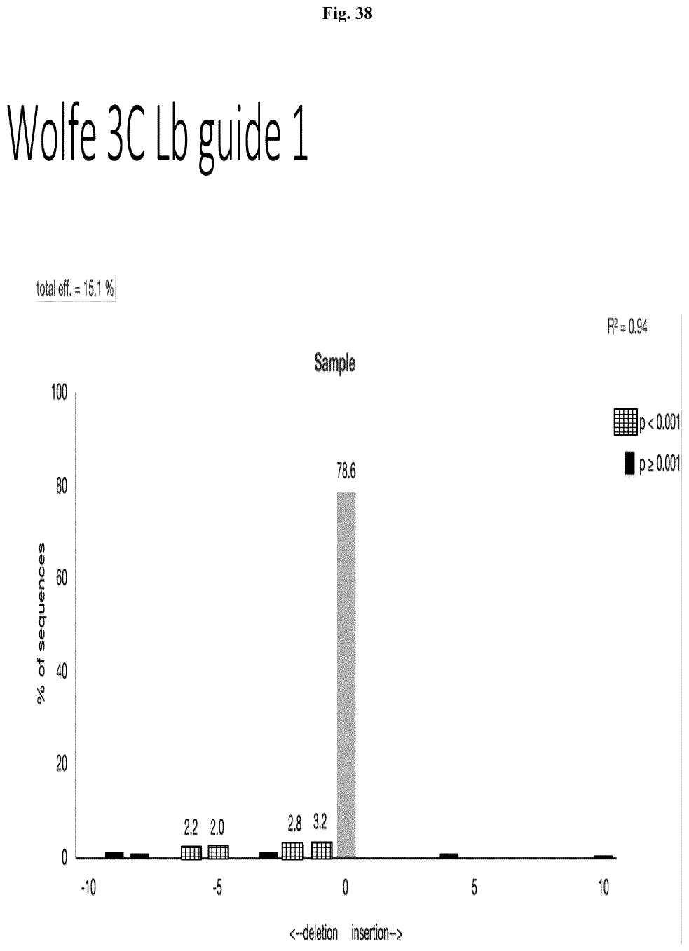

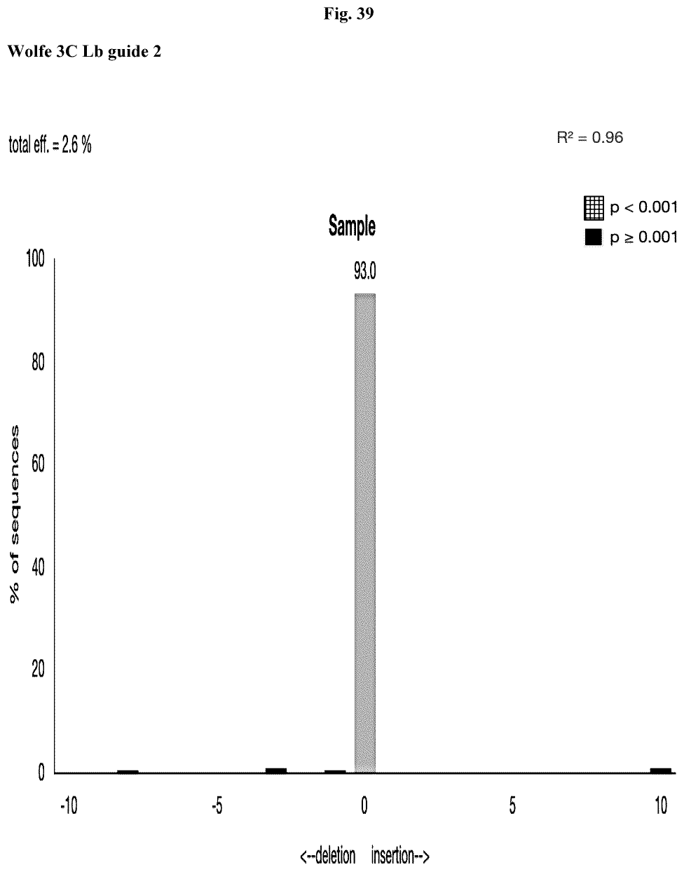

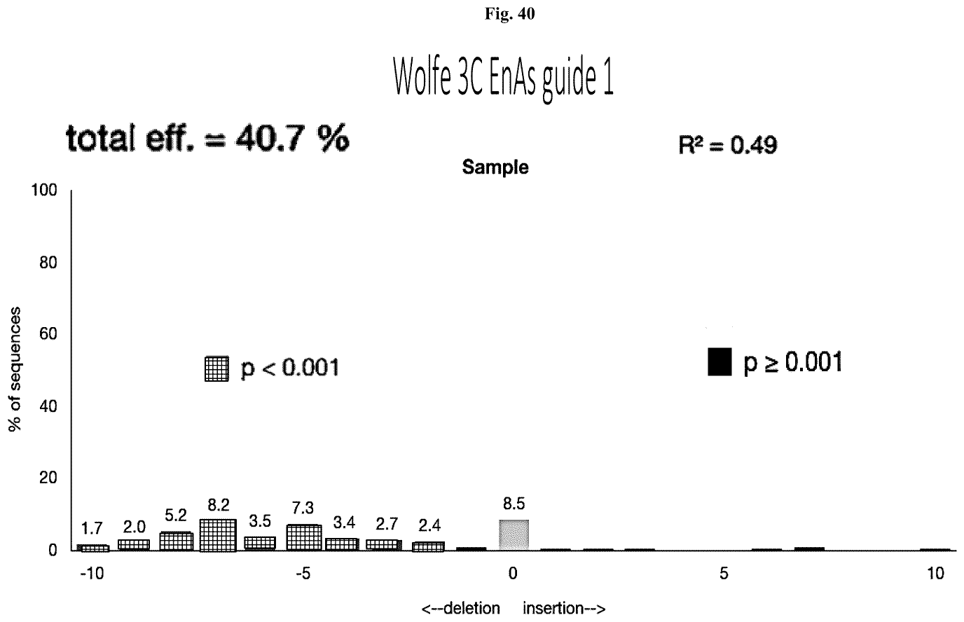

Tay-Sachs disease is caused by mutations in the HEXA gene [Fernandes Filho, J. A. & Shapiro, B. E. Tay-Sachs disease. Arch. Neurol. 61, 1466-1468 (2004).]. The most common disease allele within the patient population is a GATA microduplication within the HEXA gene. The editing efficiency of various Cas12a/sgRNA complexes was evaluated to target this microduplication to attempt to restore the wildtype sequence (create a 4 bp deletion). Cas12a RNPs (60pmol of protein) were delivered by electroporation to B-EBV cells containing a GATA microduplication in the HexA locus[GM11852 TAY-SACHS DISEASE; Coriell], or to wild-type HEXA cells to test for unwanted editing of the wild-type allele. It was found that 3×NLS-NLP-cMyc-cMyc LbaCas12a (3C-LbaCas12a) with guide 1 (FIG. 35) or guide 2 (FIG. 36) delivered as an RNP produced the desired 4 bp deletion restoring the wild-type sequence. Conversely, the 3×NLS-NLP-cMyc-cMyc enAspCas12a (3C-enAspCas12a) with guide 1 produced a number of different editing products the majority of which were not the desired sequence (FIG. 37). The 3×NLS-NLP-cMyc-cMyc LbaCas12a (3C-LbaCas12a) with guide 1 (FIG. 38) or guide 2 (FIG. 39) produced minimal editing at a wild-type HEXA sequence, whereas the 3×NLS-NLP-cMyc-cMyc enAspCas12a (3C-enAspCas12a) with guide 1 produced extensive undesired editing of the wild-type sequence. These data demonstrate the 3C-LbaCas12a can selectively repair the HEXA locus restoring the wildtype sequence for carriers of the 4 bp GATA microduplication.

### V. Genome Editing in Natural Killer Cells

Natural killer (NK) cells are subset of innate lymphoid cells (ILC) that are believed responsible for granzyme and perforin-mediated cytolytic activity against tumor and virus-infected cells [PMID: 30150991]. While NK cells are well studied, reverse genetic studies to determine the function of specific genes and application as a theraupetic agent is not understood.

Although it is not necessary to understand the mechanism of an invention, it is believed that applying conventional genetic engineering methods to NK cells encounters significant challenges. For example, retroviral transduction to deliver nucleases and shRNAs typically require high viral titer and may pose concerns of insertional mutagenesis and oncogenesis [PMID: 33463756]. Furthermore, lentiviral transduction can be inconsistent for NK cells and plasmid-based expression of genome engineering tools have been limited in efficiency. These observations have encouraged those skilled in the art to deliver RNPs into NK cells.

However, there has been limited success in geneticly engineering NK cells with Cas9 due to either lower indel rates (<80%) or low knockout efficiency [PMID: 33463756] [PMID: 33433623]. While success with Cas9 has been suboptimal, alternative approaches in engineering NK cells are constantly being investigated. dx.doi.org/10.1136/jitc-2020-SITC2020.0145.

The data presented herein show improved gene editing in NK cells with the NLS variants in transformed cells. To determine if NLS variant Cas12a nuclease platforms can facilitate gene inactivation in NK cells, 1×NLS-NLP and the 3×NLS-NLP-cMyc-cMyc (simplified as 3×NLS hereafter) enAspCas12a RNP targeting either IFNG and CD96 were delivered into NK cells via electroporation.

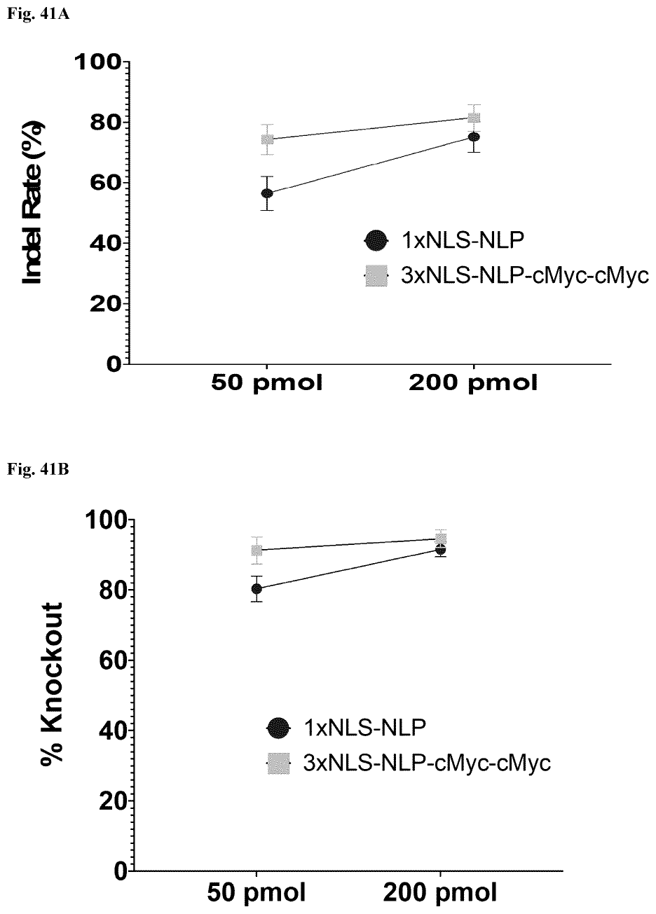

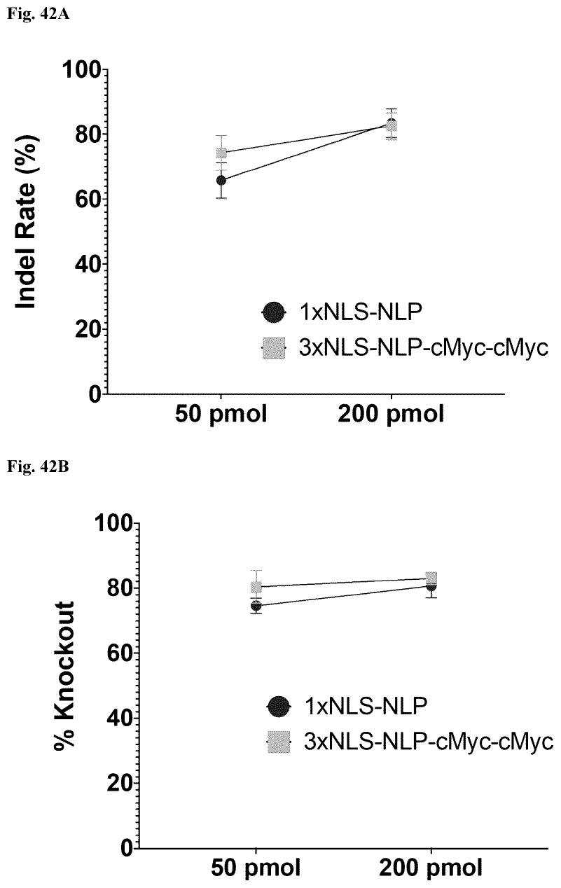

At the IFNG target site, we found that the mean indel rates for 3×NLS enAspCas12a (75.13% and 81.52%) were elevated compared to 1×NLS-NLP enAspCas12a (56.47% and 74.33%) at both 50 pmol and 200 pmol. See, FIGS. 41A and 41B. There was no observation of any cellular toxicity associated with either dosage.

At the CD96 target site, a more modest improvement in gene editing activity was observed for 3×NLS enAspCas12a (71.42% and 85.37%) as compared to 1×NLS-NLP enAspCas12a (64.84% and 80.02%) at both doses. See, FIGS. 42A and 42B. The data suggest that the impact of gene editing on protein expression is robust.

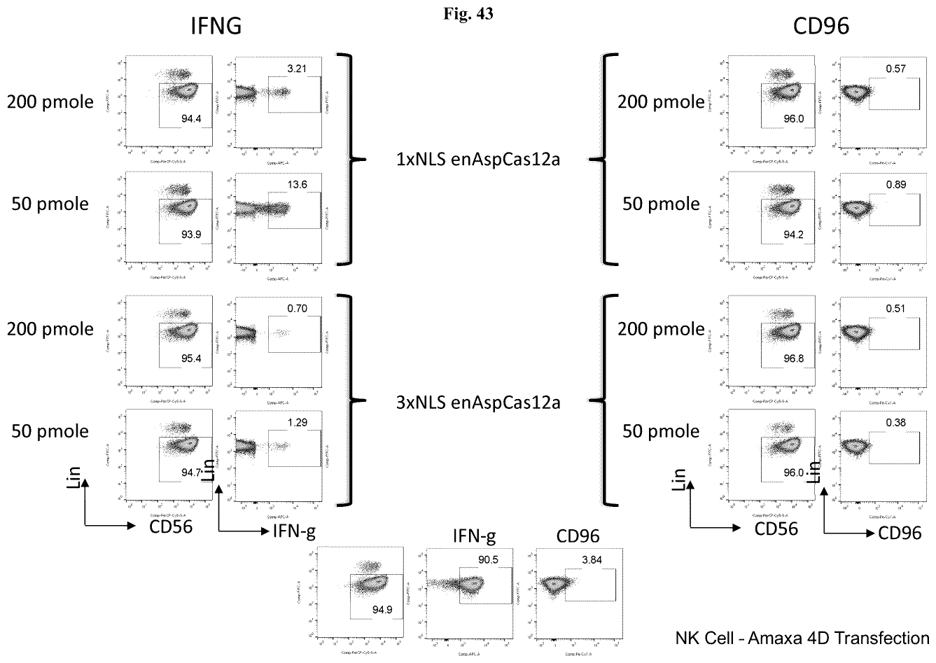

IFNg expression in NK cells can be stimulated through PMA+ionomycin or a IL-12+IL-15+IL-18 cytokine cocktail treatment. In stimulated cells, it was observed that both 1×NLS-NLP and 3×NLS enAspCas12a RNP efficiently disrupt the expression of both IFNg (% Knockout [KO]>80%). When comparing the % KO by 1×NLS-NLP and 3×NLS enAspCas12a at 50 pmol, % KO by 3×NLS enAspCas12a was increased. See, FIGS. 41 and 43. Furthermore, while only a fraction of the NK cells express CD96, % KO by 1×NLS-NLP and 3×NLS enAspCas12a of CD96 was increased by >70%. See, FIGS. 42 and 43.

Together these results demonstrate that enAspCas12a is a robust nuclease in NK cells, and that the presently disclosed NLS variant Cas12 constructs improves the editing activity and % KO efficiency of enAspCas12a in NK cells.

In NK cells, it was found that the inclusion of the additional NLSs improved the indel rates of Cas12a. Furthermore, the knockout of a relevant gene, IFNG, was improved in a 3×NLS construct. Interestingly, the improvements in activity were more apparent at the lower concentration of RNP (50 pmol RNP). At the higher concentration (200 pmol RNP), a saturation of the indel rates was observed.

The data herein show a heretofore unreported robust genome editing with RNPs in NK cells utilizing the Cas12a nuclease platform. The ability to effectively mutagenize NK cells with Cas12a nuclease may prove valuable when considering reverse genetic screens for studying the functions of NK cells or in a more therapeutic sense, the generation of chimeric antigen receptor (CAR)-engineered NK (CAR-NK) cells.

VI. 3×NLS enAspCas12a Targeting of Genetic Regulatory Elements in CD34⁺ HSPCs

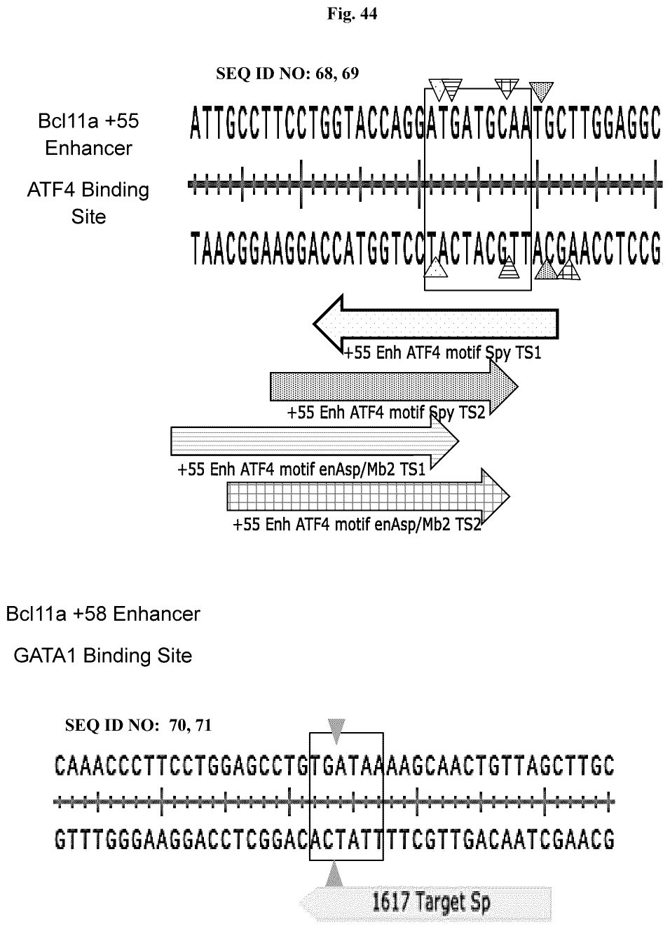

To test the presently disclosed NLS variant Cas12a nuclease platforms at a therapeutically relevant genomic locus, enAspCas12a was tested on the ATF4-binding motif within the BCL11A erythroid-lineage-specific enhancer (+55 kb) element [PMID: 32299090] [PMID: 32755585]. See, FIG. 44.

Previous studies found that disruption of this regulatory element in CD34+ HSPCs silences BCL11A expression in the erythroid lineage, which results in increased production of fetal γ-globin (HbF) protein in differentiated red blood cells [PMID: 32299090]. A similar induction of fetal γ-globin has been observed with the disruption of the GATA1-binding motif within the BCL11A erythroid-lineage-specific enhancer (+58 kb) element in CD34+ HSPCs which is currently under clinical development as an autologous bone marrow transplantation approach for the treatment of beta-hemoglobinopathies. [PMID: 28344999] and [PMID: 30911135]; see also, FIG. 44: 1617 target site.

To evaluate HbF induction in the erythroid lineage when the ATF4 site within the BCL11A +55 kb element is completely disrupted in CD34+ HSPCs, 3×NLS enAspCas12a or 3×NLS Mbo2Cas12a was utilized to target the ATF4 site in the BCL11A +55 enhancer due to the absence of a suitable TTTV PAM for AspCas12a and LbaCas12a. EnAspCas12a or Mbo2Cas12a can recognize more diverse PAM sequences, which allows their positioning for cleavage within the ATF4 target sequence. See, FIG. 44. There are also a SpyCas9 target sites with cleavage sites neighboring or overlapping the ATF4-binding motif, one of which was previously validated by others to drive HbF induction [PMID: 32299090]; SpyTS1; and FIG. 44.

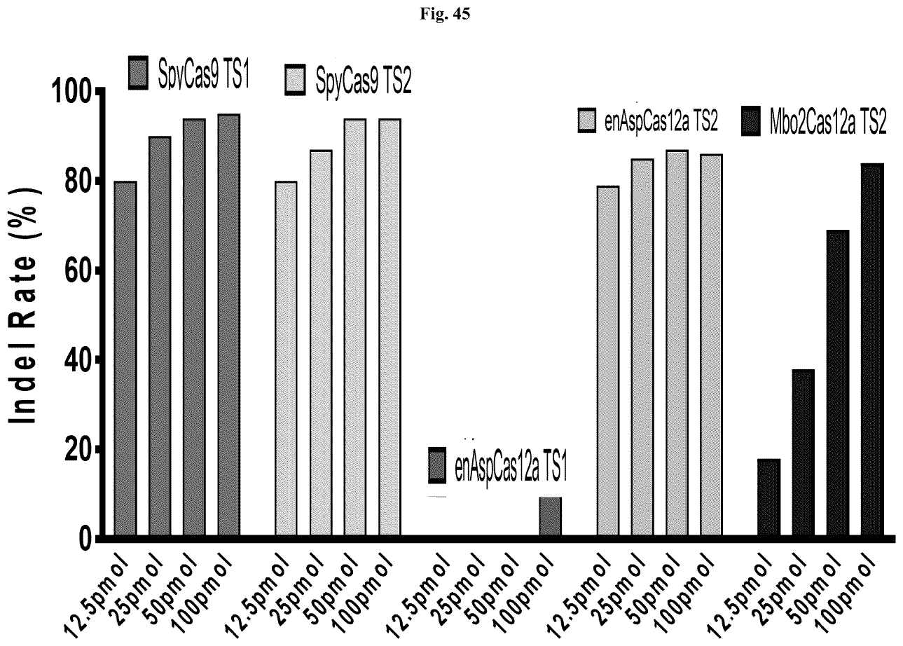

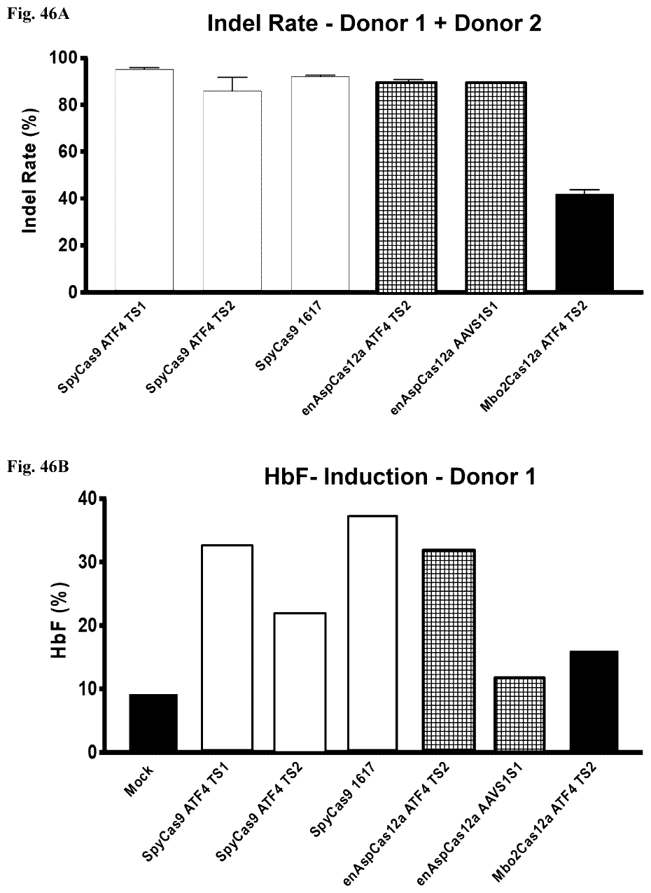

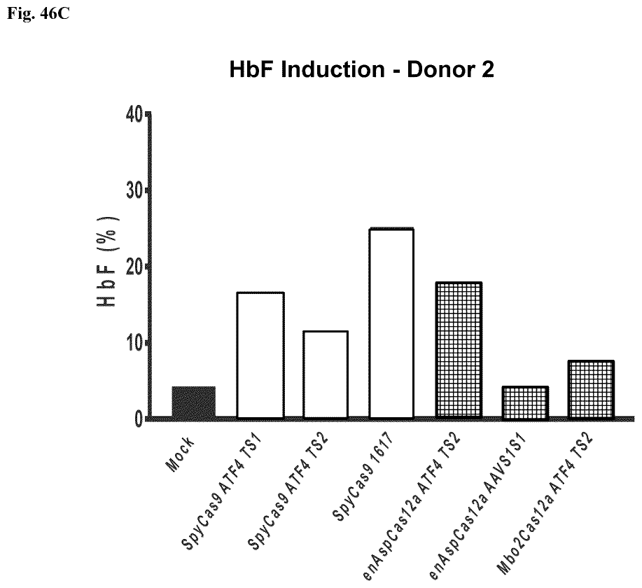

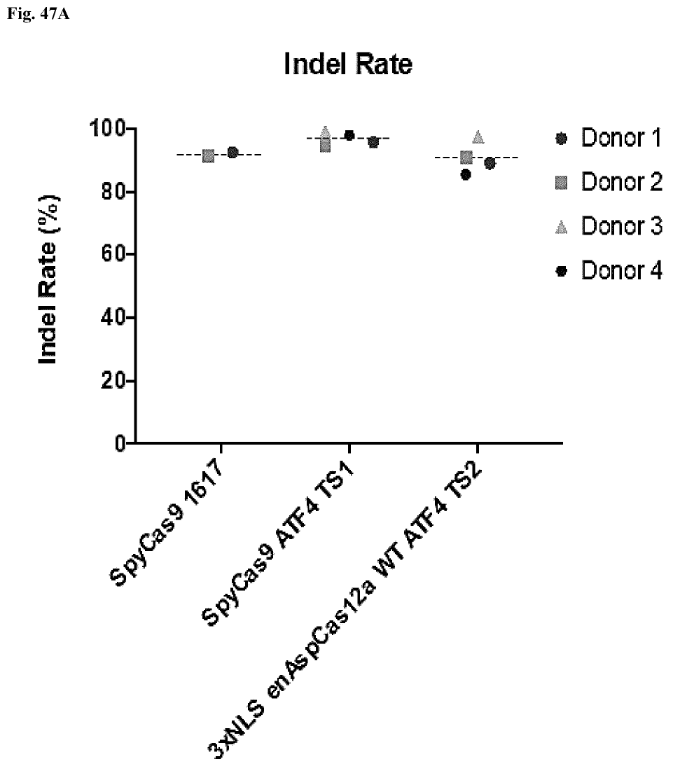

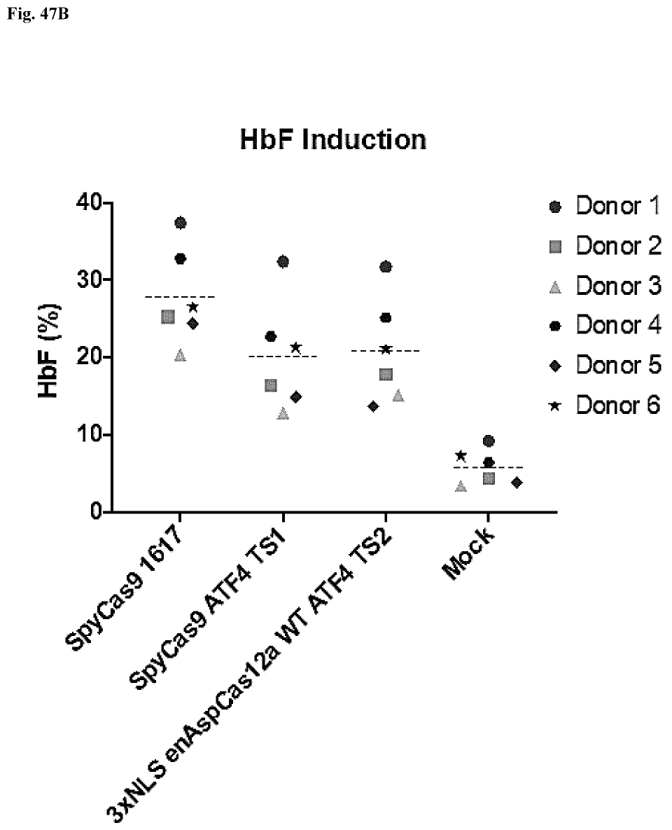

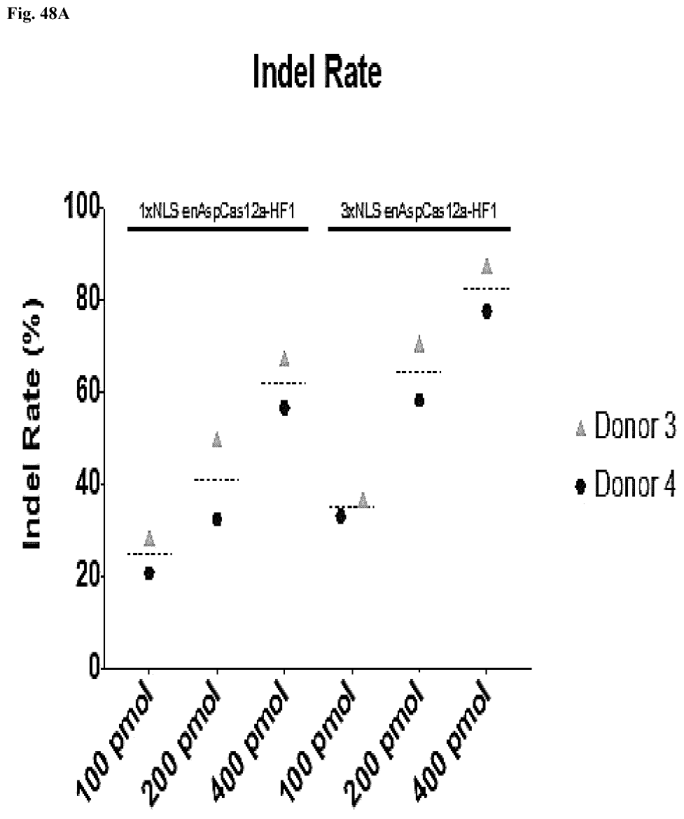

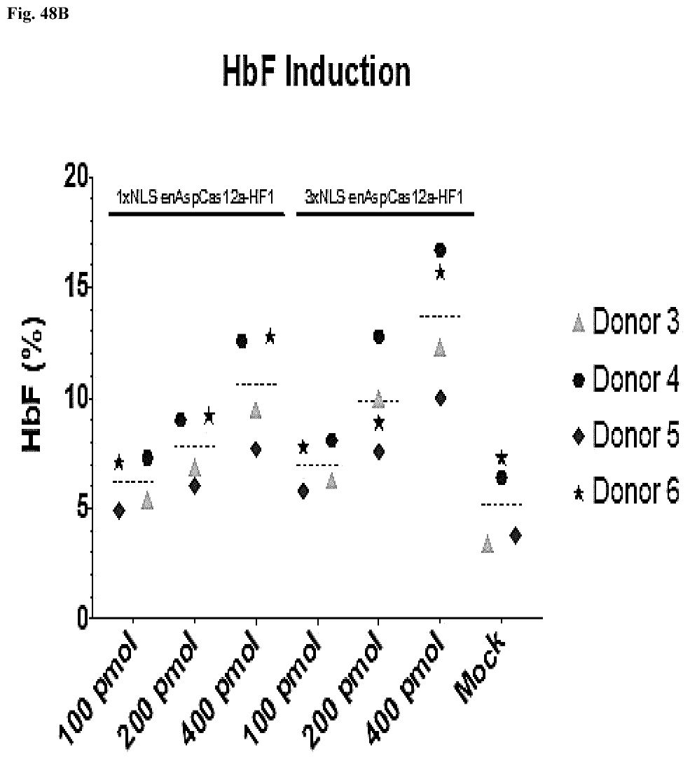

The editing rates for target site disruption was tested as a function of the Cas9 or Cas12a RNP concentration when delivered by electroporation in HEK293T cells. Four of the five RNP complexes (SpyCas9 TS1 & TS2; enAsCas12a TS2 & Mbo2Cas12a TS2) achieved a high rate of editing. See, FIG. 45.

Further experiments were performed with four active RNP complexes in CD34⁺ HSPCs from two normal donors. CD34⁺ HSPCs were electroporated with 200 pmol SpyCas9 or Cas12a complexed with their respective guide RNAs targeting the ATF4-binding motif in the +55 enhancer of BCL11a (ATF4 target sites), the GATA1-binding motif in the +58 enhancer of BCL11a (SpyCas9 1617), or a non-target control (AAVS1S1). See, FIG. 46. The data shows that all SpyCas9 and enAsCas12a RNPs were able to effectively mutagenize their target sites (indel rates >80%), but that Mbo2Cas12a had more modest activity. See, FIG. 46A. Editing of many of these target sites led to robust HbF protein expression in erythroid progenitor cells differentiated from the edited CD34+ HSPCs. See, FIGS. 46B and 46C.

Consistent with previous data, disruption of the GATA1 binding site in the BCL11A +58 enhancer by SpyCas9 1617 RNP dramatically upregulated HbF expression in erythroid progenitor cells. PMID: 30911135; and 33283989. Similar rates of HbF expression were obtained for editing in the BCL11A +55 enhancer at the ATF4 target by SpyCas9 ATF4 TS1 RNP or enAsCas12a ATF4 TS2 RNP. More modest HbF induction was obtained for the SpyCas9 ATF4 TS2 RNP. The HbF induction rates were similar for the Cas12a AAVS1S1 (control) edited and mock edited controls for these two donors.

Additional editing and HbF induction data were collected on CD34⁺ HSPCs from four additional normal donors (Donors 3-6) for SpyCas9 1617, SpyCas9 ATF4 TS1 and enAsCas12a ATF4 TS2 RNPs. Average editing rates are >85% for all of these target sites by Illumina sequencing. See, FIG. 47A. HbF induction rates for erythroid progenitors derived from edited CD34+ HSPCs are the highest for the SpCas9 1617 target site in the +58 enhancer compared to mock treated cells (27.78%, p-value <0.0001). See, FIG. 47B. Modestly lower HbF induction levels are achieved for editing in the ATF4 site by SpyCas9 TS1 (20.08%, comparison to mock p-value <0.0009) and enAspCas12a TS2 (20.75%, comparison to mock p-value <0.0005).

The efficacy of 3×NLS enAspCas12-HF1 (High-fieldity) was also evaluated for activity in human CD34+ HSPCs. A titration experiment was performed with 1×NLS-NLP and 3×NLS enAspCas12a-HF1 versions in CD34⁺ HSPCs with four different donors delivering RNPs targeting the ATF4 TS2 site by electroporation. The data show a dose-dependent editing for 1×NLS-NLP and 3×NLS enAspCas12a-HF1. See, FIG. 48A. Further, the overall editing activity of the new 3×NLS format was elevated compared to 1×NLS-NLP. Additionally, HbF levels in erythroid progenitors were elevated for 3×NLS enAspCas12a-HF1 relative to the 1×NLS-NLP, where these differences are statistically significant for the 100 pmol (p-value <0.0004) and 400 pmol (p-value <0.0042) treatment groups. See, FIG. 48B. Together, these results suggest that the Cas12a NLS variants as disclosed herein are a useful platform for the disruption of functional sequence motifs in therapeutically relevant genomic loci and that the 3×NLS architecture provides advantages for editing in primary hematopoietic cells over conventional transfection techniques.

These improvements to Cas12a via modifications to the NLS architecture produced a more robust genome editing platform in CD34+ HSPCs. Specifically, comparison between 1×NLS-NLP and 3×NLS enAspCas12a-HF1 demonstrated that there was a dose-dependent nature to the editing activity and level of HbF induction. Across four donors and three concentrations (100pmol, 200pmol, and 400pmol), editing CD34⁺ HSPCs with 3×NLS enAspCas12a-HF1 lead to a more robust HbF induction compared to 1×NLS-NLP enAspCas12a-HF1 in differentiated erythrocytes.

A comparison of editing activity and HbF induction levels in differentiated erythroid progenitors via mutagenesis of the ATF4 bind-motif at the +55 enhancer of BCL11a by either Cas9 or Cas12a to mutagenesis GATA1-binding motif within +58 kb element by Cas9 showed that while Cas9 was slightly more active than Cas12a, Cas12a editing activity saturated at ˜90%.

Furthermore, both Cas9 and Cas12a mutagenesis at the ATF4 bind-motif at the +55 enhancer of BCL11a were found to be comparable to mutagenesis GATA1-binding motif within +58 kb element by Cas9. Given the comparable levels of HbF induction by Cas12a at the ATF4 bind-motif at the +55 enhancer of BCL11a and the observation that the presently disclosed NLS variants lead to improved gene editing, these nucleases could also be particularly useful as an alternative therapeutic target for beta-hemoglobinopathies.

### EXPERIMENTAL

### Example I

### Plasmid Constructs

Cas12a nuclease experiments for neon transfection in cell culture employed the following plasmids: All AspCas12a (PMID: 26422227), enAspCas12a (PMID: 30742127), and LbaCas12a (PMID: 26422227), Mbo2Cas12a (PMID: 31723075), Mbo3Cas12a (PMID: 31723075), TspCas12a)PMID: 31723075) protein expression for protein purification utilized pET-21a protein expression plasmids (Novagen). AspCas12a, enAspCas12a, and LbaCas12a NLS variant expression constructs were constructed containing a 6×His tag at the C-terminus for affinity purification. FIG. 2.

### Example II

### Cell Culture Nuclease Assays

Human Embryonic Kidney (HEK293T) cells were cultured in high glucose DMEM with 10% FBS and 1% Penicillin/Streptomycin (Gibco) in a 37° C. incubator with 5% CO₂. K562 and Jurkat cells were cultured in RPMI 1640 medium with 10% FBS and 1% Penicillin/Streptomycin (Gibco) in a 37° C. incubator with 5% CO₂.

These cells were authenticated by University of Arizona Genetics Core and tested for mycoplasma contamination at regular intervals. For Neon transfection, early to mid-passage cells (passage number 5-15) were used. Cas12a RNPs were delivered to HEK293T, Jurkat or K562 cells by nucleofection. AspCas12a, enAspCas12a, or LbaCas12a protein were complexed with the desired crRNA at a ratio of 1:2.5 in Neon R buffer (Thermo Fisher Scientific) and incubated at room temperature (RT) for 15 minutes. For HEK293T cells, the Cas12a RNP complex was then mixed with 1×10⁵ cells in Neon R buffer at the desired concentration and electroporated using Neon® Transfection System 10 L Kit (Thermo Fisher Scientific) using the suggested electroporation parameters: Pulse voltage (1500 v), Pulse width (20 ms), Pulse number (2). For Jurkat and K562 cells, the Cas12a RNP complex was then mixed with 1×10⁵ cells in Neon R buffer at the desired concentration and electroporated using Neon® Transfection System 10 L Kit (Thermo Fisher Scientific) using the suggested electroporation parameters: Pulse voltage (1600 v), Pulse width (10 ms), Pulse number (3).

### Example III

### AspCas12a and LbaCas12a Protein Purification

Protein purification for all AspCas12a, enAspCas12a, or LbaCas12a NLS variants used a common protocol as previously described (PMID: 30892626). The plasmid expressing each Cas12a protein was introduced into E. coli Rosetta (DE3)pLysS cells (EMD Millipore) for protein overexpression. Cells were grown at 37° C. to an OD600 of ˜0.2, then shifted to 18° C. and induced for 16 hours with IPTG (1 mM final concentration).

Following induction, cells were pelleted by centrifugation and then resuspended with Nickel-NTA buffer (20 mM TRIS+1 M NaCl+20 mM imidazole+1 mM TCEP, pH 7.5) supplemented with HALT Protease Inhibitor Cocktail, EDTA-Free (100×) (ThermoFisher) and lysed with M-110s Microfluidizer (Microfluidics) following the manufacturer's instructions.

The protein was purified with Ni-NTA resin and eluted with elution buffer (20 mM TRIS, 500 mM NaCl, 250 mM Imidazole, 10% glycerol, pH 7.5). Cas12a protein was dialyzed overnight at 4° C. in 20 mM HEPES, 500 mM NaCl, 1 mM EDTA, 10% glycerol, pH 7.5. Subsequently, Cas12a protein was step dialyzed from 500 mM NaCl to 200 mM NaCl (Final dialysis buffer: 20 mM HEPES, 200 mM NaCl, 1 mM EDTA, 10% glycerol, pH 7.5). Next, the protein was purified by cation exchange chromatography (Column=5 ml HiTrap-S, Buffer A=20 mM HEPES pH 7.5+1 mM TCEP, Buffer B=20 mM HEPES pH 7.5+1 M NaCl+1 mM TCEP, Flow rate=5 ml/min, CV=column volume=5 ml).

For Cas12a variants utilized following the initial activity screen, cation exchanged chromatography was followed by size-exclusion chromatography (SEC) on Superdex-200 (16/60) column (Isocratic size-exclusion running buffer=20 mM HEPES pH 7.5, 300 mM NaCl, 1 mM TCEP). The primary protein peak from the SEC was concentrated in an Ultra-15 Centrifugal Filters Ultracel −30 K (Amicon) to a concentration of between 50 to 100 μM. The purified protein quality was assessed by SDS-PAGE/Coomassie staining to be >95% pure.

### Example IV

### Synthesis of Human Genome Specific CRISPR RNAs

Synthetic AspCas12a and LbaCas12a CRISPR RNAs (crRNAs) to AAVS1S1, EMX1S1, and DNMT1S1 were synthesized by Integrated DNA Technologies (IDT) with their proprietary modifications to each end of the crRNA (AITR1 modification on 5′ end and AITR2 modification on 3′ end):

The AspCas12a AAVS1S1 crRNA sequence is:
SEQ ID NO: 36
/AITR1/rUrArArUrUrUrCrUrArCrUrCrUrUrGr
UrArGrArUrUrCrUrGrUrCrCrCrCrUrCrCrArCr
CrCrCrArCrArGrUrG/AITR2/.
The AspCas12a EMX1S1 crRNA sequence is:
SEQ ID NO: 37
/AITR1/rUrArArUrUrUrCrUrArCrUrCrUrUrGr
UrArGrArUrUrCrArUrCrUrGrUrGrCrCrCrCrU
rCrCrCrUrCrCrCrUrG/AITR2/.
The AspCas12a DNMT1S3 crRNA sequence is:
SEQ ID NO: 38
/AITR1/rUrArArUrUrUrCrUrArCrUrCrUrUrGr
UrArGrArUrCrUrGrArUrGrGrUrCrCrArUrGrU
rCrUrGrUrUrArCrUrC/AITR2/.
The LbaCas12a AAVS1S1 crRNA sequence is:
SEQ ID NO: 39
/AITR1/rUrArArUrUrUrCrUrArCrUrArArGrUr
GrUrArGrArUrUrCrUrGrUrCrCrCrCrUrCrCrA
rCrCrCrCrArCrArGrUrG/AITR2/.
The LbaCas12a EMX1S1 crRNA sequence is:
SEQ ID NO: 40
/AITR1/rUrArArUrUrUrCrUrArCrUrArArGrUr
GrUrArGrArUrUrCrArUrCrUrGrUrGrCrCrCrC
rUrCrCrCrUrCrCrCrUrG/AITR2/.
The LbaCas12a DNMT1S3 crRNA sequence is:
SEQ ID NO: 41
/AITR1/rUrArArUrUrUrCrUrArCrUrArArGrUrGr
UrArGrArUrCrUrGrArUrGrGrUrCrCrArUrG
rUrCrUrGrUrUrArCrUrC/AITR2/.

### Example V

### Target Site Indel Frequency Analysis in Mammalian Cells By Deep Sequencing

Library construction for deep sequencing is modified from published protocols (PMID: 26480473; and PMID: 30892626).

For analysis of mammalian cell culture experiments, cells were harvested 72 h after transfection (or nucleofection) and genomic DNA extracted with GenElute Mammalian Genomic DNA Miniprep Kit (Sigma). Briefly, regions flanking each target site were PCR amplified using locus-specific primers bearing tails complementary to the Truseq adapters as described previously. 25-50 ng input genomic DNA is PCR amplified with Q5 High Fidelity DNA Polymerase (New England Biolabs): (98° C., 15s; 65° C. 30s; 72° C. 30s)×30 cycles. 1 μl of each PCR reaction was amplified with barcoded primers to reconstitute the TruSeq adaptors using the Phusion High Fidelity DNA Polymerase (New England Biolabs): (98° C., 15s; 64° C., 25s; 72° C., 25s)×10 cycles. Equal amounts of the products were pooled and gel purified. The purified library was deep sequenced using a paired-end 150 bp Illumina MiniSeq run.

MiniSeq data analysis was performed using a suite of Unix-based software tools. First, the quality of paired-end sequencing reads (R1 and R2 fastq files) was assessed using FastQC. Raw paired-end reads were combined using paired end read merger (PEAR) to generate single merged high-quality full-length reads. Reads were then filtered by quality (using Filter FASTQ) to remove those with a mean PHRED quality score under 30 and a minimum per base score under 24. Each group of reads was then aligned to a corresponding reference sequence using BWA (version 0.7.5) and SAMtools (version 0.1.19).

To determine indel frequency, size and distribution, all edited reads from each experimental replicate were combined and aligned, as described above. Indel types and frequencies were then cataloged in a text output format at each base using bam-readcount (github.com/genome/bam-readcount). For each treatment group, the average background indel frequencies (based on indel type, position and frequency) of the triplicate negative control group were subtracted to obtain the nuclease-dependent indel frequencies.

We claim:

1. A Cas12a fusion protein comprising a Cas12a protein, at least one c-Myc nuclear localization signal sequence and a nucleoplasmin nuclear localization signal sequence.

2. The Cas12a fusion protein of claim 1, wherein a C-terminal portion of said Cas12a fusion protein comprises said at least one c-Myc nuclear localization signal sequence.

3. The Cas12a fusion protein of claim 1, wherein an N-terminal portion of said Cas12a fusion protein comprises said at least one c-Myc nuclear localization signal sequence.

4. The Cas12a fusion protein of claim 1, wherein said C-terminal portion of said Cas12a fusion protein comprises said nucleoplasmin nuclear localization signal sequence.

5. The Cas12a fusion protein of claim 1, wherein said Cas12a protein is selected from the group consisting of an Acidaminococcus sp Cas12a protein (AspCas12a or enAspCas12a), a Lachnospiraceae bacterium Cas12a protein (LbaCas12a), Moraxella bovoculi AAX08_00205 Cas12a protein (Mbo2Cas12a), Moraxella bovoculi AAX11_00205 Cas12a protein (Mbo3Cas12a), Thiomicrospira sp Cas12a protein (TspCas12a) and Francisella novicida U112 Cas12a protein.

6. The Cas12a fusion protein of claim 1, further comprising an SV40 nuclear localization signal sequence.

7. The Cas12a fusion protein of claim 6, wherein said SV40 nuclear localization signal sequence is a BP SV40 nuclear localization signal sequence.

8. The Cas12a fusion protein of claim 6, wherein said SV40 nuclear localization signal sequence is a large T antigen SV40 nuclear localization signal sequence.

9. The Cas12a fusion protein of claim 1, further comprising at least two c-Myc nuclear localization signal sequences.

10. The Cas12a fusion protein of claim 9, wherein a C-terminal portion of said Cas12a fusion protein comprises said at least two c-Myc nuclear localization signal sequences.

11. The Cas12a fusion protein of claim 9, wherein said N-terminal portion of said Cas12a fusion protein comprises said at least two c-Myc nuclear localization signal sequence.

12. The Cas12a fusion protein of claim 1, further comprising at least three c-Myc nuclear localization signal sequences.

13. The Cas12a fusion protein of claim 9, wherein a C-terminal portion of said Cas12a fusion protein comprises one of said at least three c-Myc nuclear localization sequences and an N-terminal portion of said Cas12 fusion protein comprises two of said at least three c-Myc nuclear localization signal sequences.

14. The Cas12a fusion protein of claim 1, wherein a C-terminal portion of said Cas12a fusion protein comprises said SV40 nuclear localization signal sequence.

15. The Cas12a fusion protein of claim 1, wherein said N-terminal portion of said Cas12a fusion protein comprises said SV40 nuclear localization signal sequence.

16. A method, comprising:
a) providing;
i) a patient exhibiting at least one symptom of a genetic disease;
ii) a pharmaceutically acceptable composition comprising a Cas12a fusion protein comprising a Cas12a protein, at least one c-Myc nuclear localization signal sequence and a nucleoplasmin nuclear localization signal sequence and a carrier;
b) administering said pharmaceutically acceptable composition to said patient under conditions such that said at least one symptom of said genetic disease is reduced.

17. The method of claim 16, wherein said patient further comprises a mutated gene.

18. The method of claim 17, wherein said administering further comprises gene editing wherein said mutated gene is deleted.

19. The method of claim 17, wherein said administering further comprises gene editing wherein said mutated gene is converted to a wild type gene.

20. The method of claim 17, wherein said administering further comprises gene editing wherein said mutated gene is altered to repair its function.

21. The method of claim 17, wherein said administering further comprises gene editing wherein said mutated gene is inactivated.

22. The method of claim 16, wherein a C-terminal portion of said Cas12a fusion protein comprises said at least one c-Myc nuclear localization signal sequence.

23. The method of claim 16, wherein an N-terminal portion of said Cas12a fusion protein comprises said at least one c-Myc nuclear localization signal sequence.

24. The method of claim 16, wherein said C-terminal portion of said Cas12a fusion protein comprises said nucleoplasmin nuclear localization signal sequence.

25. The method of claim 16, wherein said Cas12a protein is selected from the group consisting of an Acidaminococcus sp Cas12a protein (AspCas12a or enAspCas12a), a Lachnospiraceae bacterium Cas12a protein (LbaCas12a), Moraxella bovoculi AAX08_00205 Cas12a protein (Mbo2Cas12a), Moraxella bovoculi AAX11_00205 Cas12a protein (Mbo3Cas12a), Thiomicrospira sp Cas12a protein (TspCas12a) and Francisella novicida U112 Cas12a protein.
